SEQUENCE LISTING (23 sequences)

SEQ ID NO: 1 - DNA, 1859 bp, Homo sapiens
gcaggcacaaactcatccatccccagttgattggaagaaacaacgatgactcctgggaagacctcattggtgtcactgctactgctgctgagcctggaggccatagtgaaggcaggaatcacaatcccacgaaatccaggatgcccaaattctgaggacaagaacttcccccggactgtgatggtcaacctgaacatccataaccggaataccaataccaatcccaaaaggtcctcagattactacaaccgatccacctcaccttggaatctccaccgcaatgaggaccctgagagatatccctctgtgatctgggaggcaaagtgccgccacttgggctgcatcaacgctgatgggaacgtggactaccacatgaactctgtccccatccagcaagagatcctggtcctgcgcagggagcctccacactgccccaactccttccggctggagaagatactggtgtccgtgggctgcacctgtgtcaccccgattgtccaccatgtggcctaagagctctggggagcccacactccccaaagcagttagactatggagagccgacccagcccctcaggaaccctcatccttcaaagacagcctcatttcggactaaactcattagagttcttaaggcagtttgtccaattaaagcttcagaggtaacacttggccaagatatgagatctgaattacctttccctctttccaagaaggaaggtttgactgagtaccaatttgcttcttgtttacttttttaagggctttaagttatttatgtatttaatatgccctgagataactttggggtataagattccattttaatgaattacctactttattttgtttgtctttttaaagaagataagattctgggcttgggaattttattatttaaaaggtaaaacctgtatttatttgagctatttaaggatctatttatgtttaagtatttagaaaaaggtgaaaaagcactattatcagttctgcctaggtaaatgtaagatagaattaaatggcagtgcaaaatttctgagtctttacaacatacggatatagtatttcctcctctttgtttttaaaagttataacatggctgaaaagaaagattaaacctactttcatatgtattaatttaaattttgcaatttgttgaggttttacaagagatacagcaagtctaactctctgttccattaaacccttataataaaatccttctgtaataataaagtttcaaaagaaaatgtttatttgttctcattaaatgtattttagcaaactcagctcttccctattgggaagagttatgcaaattctcctataagcaaaacaaagcatgtctttgagtaacaatgacctggaaatacccaaaattccaagttctcgatttcacatgccttcaagactgaacaccgactaaggttttcatactattagccaatgctgtagacagaagcattttgataggaatagagcaaataagataatggccctgaggaatggcatgtcattattaaagatcatatggggaaaatgaaaccctccccaaaatacaagaagttctgggaggagacattgtcttcagactacaatgtccagtttctcccctagactcaggcttcctttggagattaaggcccctcagagatcaacagaccaacatttttctcttcctcaagcaacactcctagggcctggcttctgtctgatcaaggcaccacacaacccagaaaggagctgatggggcagaacgaactttaagtatgagaaaagttcagcccaagtaaaataaaaactcaatcacattcaattccagagtagtttcaagtttcacatcgtaaccattttcgccc

SEQ ID NO: 2 - protein, 155 aa, Homo sapiens
MTPGKTSLVSLLLLLSLEAIVKAGITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 3 - DNA, 687 bp, Homo sapiens
aggcgggcagcagctgcaggctgaccttgcagcttggcggaatggactggcctcacaacctgctgtttcttcttaccatttccatcttcctggggctgggccagcccaggagccccaaaagcaagaggaaggggcaagggcggcctgggcccctggcccctggccctcaccaggtgccactggacctggtgtcacggatgaaaccgtatgcccgcatggaggagtatgagaggaacatcgaggagatggtggcccagctgaggaacagctcagagctggcccagagaaagtgtgaggtcaacttgcagctgtggatgtccaacaagaggagcctgtctccctggggctacagcatcaaccacgaccccagccgtatccccgtggacctgccggaggcacggtgcctgtgtctgggctgtgtgaaccccttcaccatgcaggaggaccgcagcatggtgagcgtgccggtgttcagccaggttcctgtgcgccgccgcctctgcccgccaccgccccgcacagggccttgccgccagcgcgcagtcatggagaccatcgctgtgggctgcacctgcatcttctgaatcacctggcccagaagccaggccagcagcccgagaccatcctccttgcacctttgtgccaagaaaggcctatgaaaagtaaacactgacttttgaaagcaag

SEQ ID NO: 4 - protein, 180 aa, Homo sapiens
MDWPHNLLFLLTISIFLGLGQPRSPKSKRKGQGRPGPLAPGPHQVPLDLVSRMKPYARMEEYERNIEEMVAQLRNSSELAQRKCEVNLQLWMSNKRSLSPWGYSINHDPSRIPVDLPEARCLCLGCVNPFTMQEDRSMVSVPVFSQVPVRRRLCPPPPRTGPCRQRAVMETIAVGCTCIF

SEQ ID NO: 5 - DNA, 1048 bp, Homo sapiens
gccaggtgtgcaggccgctccaagcccagcctgccccgctgccgccaccatgacgctcctccccggcctcctgtttctgacctggctgcacacatgcctggcccaccatgacccctccctcagggggcacccccacagtcacggtaccccacactgctactcggctgaggaactgcccctcggccaggcccccccacacctgctggctcgaggtgccaagtgggggcaggctttgcctgtagccctggtgtccagcctggaggcagcaagccacagggggaggcacgagaggccctcagctacgacccagtgcccggtgctgcggccggaggaggtgttggaggcagacacccaccagcgctccatctcaccctggagataccgtgtggacacggatgaggaccgctatccacagaagctggccttcgccgagtgcctgtgcagaggctgtatcgatgcacggacgggccgcgagacagctgcgctcaactccgtgcggctgctccagagcctgctggtgctgcgccgccggccctgctcccgcgacggctcggggctccccacacctggggcctttgccttccacaccgagttcatccacgtccccgtcggctgcacctgcgtgctgccccgttcagtgtgaccgccgaggccgtggggcccctagactggacacgtgtgctccccagagggcaccccctatttatgtgtatttattgttatttatatgcctcccccaacactacccttggggtctgggcattccccgtgtctggaggacagccccccactgttctcctcatctccagcctcagtagttgggggtagaaggagctcagcacctcttccagcccttaaagctgcagaaaaggtgtcacacggctgcctgtaccttggctccctgtcctgctcccggcttcccttaccctatcactggcctcaggcccccgcaggctgcctcttcccaacctccttggaagtacccctgtttcttaaacaattatttaagtgtacgtgtattattaaactgatgaacacatccccaaaa

SEQ ID NO: 6 - protein, 197 aa, Homo sapiens
MTLLPGLLFLTWLHTCLAHHDPSLRGHPHSHGTPHCYSAEELPLGQAPPHLLARGAKWGQALPVALVSSLEAASHRGRHERPSATTQCPVLRPEEVLEADTHQRSISPWRYRVDTDEDRYPQKLAFAECLCRGCIDARTGRETAALNSVRLLQSLLVLRRRPCSRDGSGLPTPGAFAFHTEFIHVPVGCTCVLPRSV

SEQ ID NO: 7 - DNA, 1873 bp, Homo sapiens
aaaatgttttcagctcctggaggcgaaaggtgcagagtcgctctgtgtccgtgaggccgggcggcgacctcgctcagtcggcttctcggtccgagtccccgggtctggatgctggtagccggcttcctgctggcgctgccgccgagctgggccgcgggcgccccgagggcgggcaggcgccccgcgcggccgcggggctgcgcggaccggccggaggagctactggagcagctgtacgggcgcctggcggccggcgtgctcagtgccttccaccacacgctgcagctggggccgcgtgagcaggcgcgcaacgcgagctgcccggcagggggcaggcccgccgaccgccgcttccggccgcccaccaacctgcgcagcgtgtcgccctgggcctacagaatctcctacgacccggcgaggtaccccaggtacctgcctgaagcctactgcctgtgccggggctgcctgaccgggctgttcggcgaggaggacgtgcgcttccgcagcgcccctgtctacatgcccaccgtcgtcctgcgccgcacccccgcctgcgccggcggccgttccgtctacaccgaggcctacgtcaccatccccgtgggctgcacctgcgtccccgagccggagaaggacgcagacagcatcaactccagcatcgacaaacagggcgccaagctcctgctgggccccaacgacgcgcccgctggcccctgaggccggtcctgccccgggaggtctccccggcccgcatcccgaggcgcccaagctggagccgcctggagggctcggtcggcgacctctgaagagagtgcaccgagcaaaccaagtgccggagcaccagcgccgcctttccatggagactcgtaagcagcttcatctgacacgggcatccctggcttgcttttagctacaagcaagcagcgtggctggaagctgatgggaaacgacccggcacgggcatcctgtgtgcggcccgcatggagggtttggaaaagttcacggaggctccctgaggagcctctcagatcggctgctgcgggtgcagggcgtgactcaccgctgggtgcttgccaaagagatagggacgcatatgctttttaaagcaatctaaaaataataataagtatagcgactatatacctacttttaaaatcaactgttttgaatagaggcagagctattttatattatcaaatgagagctactctgttacatttcttaacatataaacatcgttttttacttcttctggtagaattttttaaagcataattggaatccttggataaattttgtagctggtacactctggcctgggtctctgaattcagcctgtcaccgatggctgactgatgaaatggacacgtctcatctgacccactcttccttccactgaaggtcttcacgggcctccaggtggaccaaagggatgcacaggcggctcgcatgccccagggccagctaagagttccaaagatctcagatttggttttagtcatgaatacataaacagtctcaaactcgcacaattttttcccccttttgaaagccactggggccaatttgtggttaagaggtggtgagataagaagtggaacgtgacatctttgccagttgtcagaagaatccaagcaggtattggcttagttgtaagggctttaggatcaggctgaatatgaggacaaagtgggccacgttagcatctgcagagatcaatctggaggcttctgtttctgcattctgccacgagagctaggtccttgatcttttctttagattgaaagtctgtctctgaacacaattatttgtaaaagttagtagttcttttttaaatcattaaaagaggcttgctgaaggaaaaaaaaaaaaa

SEQ ID NO: 8 - protein, 202 aa, Homo sapiens
MLVAGFLLALPPSWAAGAPRAGRRPARPRGCADRPEELLEQLYGRLAAGVLSAFHHTLQLGPREQARNASCPAGGRPADRRFRPPTNLRSVSPWAYRISYDPARYPRYLPEAYCLCRGCLTGLFGEEDVRFRSAPVYMPTVVLRRTPACAGGRSVYTEAYVTIPVGCTCVPEPEKDADSINSSIDKQGAKLLLGPNDAPAGP

SEQ ID NO: 9 - DNA, 1335 bp, Homo sapiens
ggcttgctgaaaataaaatcaggactcctaacctgctccagtcagcctgcttccacgaggcctgtcagtcagtgcccgacttgtgactgagtgtgcagtgcccagcatgtaccaggtcagtgcagagggctgcctgagggctgtgctgagagggagaggagcagagatgctgctgagggtggagggaggccaagctgccaggtttggggctgggggccaagtggagtgagaaactgggatcccagggggagggtgcagatgagggagcgacccagattaggtgaggacagttctctcattagccttttcctacaggtggttgcattcttggcaatggtcatgggaacccacacctacagccactggcccagctgctgccccagcaaagggcaggacacctctgaggagctgctgaggtggagcactgtgcctgtgcctcccctagagcctgctaggcccaaccgccacccagagtcctgtagggccagtgaagatggacccctcaacagcagggccatctccccctggagatatgagttggacagagacttgaaccggctcccccaggacctgtaccacgcccgttgcctgtgcccgcactgcgtcagcctacagacaggctcccacatggacccccggggcaactcggagctgctctaccacaaccagactgtcttctacaggcggccatgccatggcgagaagggcacccacaagggctactgcctggagcgcaggctgtaccgtgtttccttagcttgtgtgtgtgtgcggccccgtgtgatgggctagccggacctgctggaggctggtccctttttgggaaacctggagccaggtgtacaaccacttgccatgaagggccaggatgcccagatgcttggcccctgtgaagtgctgtctggagcagcaggatcccgggacaggatggggggctttggggaaaacctgcacttctgcacattttgaaaagagcagctgctgcttagggccgccggaagctggtgtcctgtcattttctctcaggaaaggttttcaaagttctgcccatttctggaggccaccactcctgtctcttcctcttttcccatcccctgctaccctggcccagcacaggcactttctagatatttcccccttgctggagaagaaagagcccctggttttatttgtttgtttactcatcactcagtgagcatctactttgggtgcattctagtgtagttactagtcttttgacatggatgattctgaggaggaagctgttattgaatgtatagagatttatccaaataaatatctttatttaaaaatgaaaaaaaaaaaaaaaaaaaa

SEQ ID NO: 10 - protein, 177 aa, Homo sapiens
MRERPRLGEDSSLISLFLQVVAFLAMVMGTHTYSHWPSCCPSKGQDTSEELLRWSTVPVPPLEPARPNRHPESCRASEDGPLNSRAISPWRYELDRDLNRLPQDLYHARCLCPHCVSLQTGSHMDPRGNSELLYHNQTVFYRRPCHGEKGTHKGYCLERRLYRVSLACVCVRPRVMG

SEQ ID NO: 11 - DNA, 808 bp, Homo sapiens
gaacacaggcatacacaggaagatacattcacagaaagagcttcctgcacaaagtaagccaccagcgcaacatgacagtgaagaccctgcatggcccagccatggtcaagtacttgctgctgtcgatattggggcttgcctttctgagtgaggcggcagctcggaaaatccccaaagtaggacatacttttttccaaaagcctgagagttgcccgcctgtgccaggaggtagtatgaagcttgacattggcatcatcaatgaaaaccagcgcgtttccatgtcacgtaacatcgagagccgctccacctccccctggaattacactgtcacttgggaccccaaccggtacccctcggaagttgtacaggcccagtgtaggaacttgggctgcatcaatgctcaaggaaaggaagacatctccatgaattccgttcccatccagcaagagaccctggtcgtccggaggaagcaccaaggctgctctgtttctttccagttggagaaggtgctggtgactgttggctgcacctgcgtcacccctgtcatccaccatgtgcagtaagaggtgcatatccactcagctgaagaagctgtagaaatgccactccttacccagtgctctgcaacaagtcctgtctgacccccaattccctccacttcacaggactcttaataagacctgcacggatggaaacagaaaatattcacaatgtatgtgtgtatgtactacactttatatttgatatctaaaatgttaggagaaaaattaatatattcagtgctaatataataaagtattaataattt

SEQ ID NO: 12 - protein, 163 aa, Homo sapiens
MTVKTLHGPAMVKYLLLSILGLAFLSEAAARKIPKVGHTFFQKPESCPPVPGGSMKLDIGIINENQRVSMSRNIESRSTSPWNYTVTWDPNRYPSEVVQAQCRNLGCINAQGKEDISMNSVPIQQETLVVRRKHQGCSVSFQLEKVLVTVGCTCVTPVIHHVQ

SEQ ID NO: 13 - DNA, 947 bp, Homo sapiens
ggcttcagttactagctaggctactgagtttagttctcagtttggcaccttgatacctttaggtgtgagtgttcccatttccaggtgaggaactgaggtgcaaagagaagccctgatcccataaaaggacaggaatgctgagttccgccagaccatgcatctcttgctagtaggtgaggcgagtctctaactgattgcagcgtcttctattttccaggtcaagtacttgctgctgtcgatattggggcttgcctttctgagtgaggcggcagctcggaaaatccccaaagtaggacatacttttttccaaaagcctgagagttgcccgcctgtgccaggaggtagtatgaagcttgacattggcatcatcaatgaaaaccagcgcgtttccatgtcacgtaacatcgagagccgctccacctccccctggaattacactgtcacttgggaccccaaccggtacccctcggaagttgtacaggcccagtgtaggaacttgggctgcatcaatgctcaaggaaaggaagacatctccatgaattccgttcccatccagcaagagaccctggtcgtccggaggaagcaccaaggctgctctgtttctttccagttggagaaggtgctggtgactgttggctgcacctgcgtcacccctgtcatccaccatgtgcagtaagaggtgcatatccactcagctgaagaagctgtagaaatgccactccttacccagtgctctgcaacaagtcctgtctgacccccaattccctccacttcacaggactcttaataagacctgcacggatggaaacataaaatattcacaatgtatgtgtgtatgtactacactttatatttgatatctaaaatgttaggagaaaaattaatatattcagtgctaatataataaagtattaataatgttaaaaaaaaaaaaaaaaaaaaaaaaa

SEQ ID NO: 14 - protein, 109 aa, Homo sapiens
MKLDIGIINENQRVSMSRNIESRSTSPWNYTVTWDPNRYPSEVVQAQCRNLGCINAQGKEDISMNSVPIQQETLVVRRKHQGCSVSFQLEKVLVTVGCTCVTPVIHHVQ

SEQ ID NO: 15 - DNA, 357 bp, Homo sapiens
caggtgcagctggtgcagtctggggctgaggtgaagaagcctggggcctcagtgaaggtttcctgcaaggtttccggatacaccctcactgagttcgccatgcactgggtgcgacaggctcctggaaaagggcttgagtggatgggaggttttgttcctgaagatggtgagacaatctacgcgcagaagttccagggcagagtcaccatgaccgaggacacatctacagacacagcctacatggagctgagcagcctgagatctgaggacacggccgtgtattactgtgcaacagatcccctgtatgagggttcgttttctgtttgggggcaggggaccacggtcaccgtctcgagt

SEQ ID NO: 16 - protein, 119 aa, Homo sapiens
QVQLVQSGAEVKKPGASVKVSCKVSGYTLTEFAMHWVRQAPGKGLEWMGGFVPEDGETIYAQKFQGRVTMTEDTSTDTAYMELSSLRSEDTAVYYCATDPLYEGSFSVWGQGTTVTVSS

SEQ ID NO: 17 - DNA, 324 bp, Homo sapiens
tcctatgtgctgactcagccaccctcggtgtcagtggccccaggacagacggccaggattacctgtgggggaaacaacattgaaagtaaaagtgtgcactggtaccagcagaagccaggccaggcccctgtgctggtggtctatgatgatagcgaccggccctcagggatccctgagcgattctctggctccaactctgggaacacggccaccctgaccatcagcagggtcgaagccggggatgaggccgactattactgtcaggtgtgggatagtaatactgatcattgggtgttcggcggagggaccaagctcaccgtccta

SEQ ID NO: 18 - protein, 108 aa, Homo sapiens
SYVLTQPPSVSVAPGQTARITCGGNNIESKSVHWYQQKPGQAPVLVVYDDSDRPSGIPERFSGSNSGNTATLTISRVEAGDEADYYCQVWDSNTDHWVFGGGTKLTVL

SEQ ID NO: 19 - protein, 449 aa, Homo sapiens
QVQLVQSGAEVKKPGASVKVSCKVSGYTLTEFAMHWVRQAPGKGLEWMGGFVPEDGETIYAQKFQGRVTMTEDTSTDTAYMELSSLRSEDTAVYYCATDPLYEGSFSVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 214 aa, Homo sapiens
SYVLTQPPSVSVAPGQTARITCGGNNIESKSVHWYQQKPGQAPVLVVYDDSDRPSGIPERFSGSNSGNTATLTISRVEAGDEADYYCQVWDSNTDHWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 21 - DNA, 11969 bp, Artificial Sequence
  synthesized expression vector
gaattcattgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctggcggccgcgacctgcaggcgcagaactggtaggtatggaagatccctcgagatccattgtgctggcggtaggcgagcagcgcctgcctgaagctgcgggcattcccagtcagaaatgagcgccagtcgtcgtcggctctcggcaccgaagtgctatgattctccgccagcatggcttcggccagtgcgtcgagcagcgcccgcttgttcctgaagtgccagtaaagcgccggctgctgaacccccaaccgttccgccagtttgcgtgtcgtcagaccgtctacgccgacctcgttcaacaggtccagggcggcacggatcactgtattcggctgcaactttgtcatgcttgacactttatcactgataaacataatatgtccaccaacttatcagtgataaagaatccgcgccagcacaatggatctcgaggtcgagggatctctagaggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgacctcgggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctgatggctctttgcggcacccatcgttcgtaatgttccgtggcaccgaggacaaccctcaagagaaaatgtaatcacactggctcaccttcgggtgggcctttctgcgtttataaggagacactttatgtttaagaaggttggtaaattccttgcggctttggcagccaagctagatccagctttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagccaagctagcttgaattaattcccgagcccttccaatacaaaaactaattagactttgagtgatcttgagcctttcctagtttttgtattggaagggctcgtcgccagtctcattgagaaggcatgtgcggacgatggcttctgtcactgcaaaggggtcacaattggcagaggggcggcggtcttcaaagtaacctttcttctcctggccgagccgagaatgggagtagagccgactgcttgattcccacaccaatctcctcgccgctctcacttcgcctcgttctcgtggctcgtggccctgtccaccccgtccatcatcccgccggccaccgctcagagcaccttccaccatggccacctcagcaagttcccacttgaacaaaaacatcaagcaaatgtacttgtgcctgccccagggtgagaaagtccaagccatgtatatctgggttgatggtactggagaaggactgcgctgcaaaacccgcaccctggactgtgagcccaagtgtgtagaagagttacctgagtggaattttgatggctctagtacctttcagtctgagggctccaacagtgacatgtatctcagccctgttgccatgtttcgggaccccttccgcagagatcccaacaagctggtgttctgtgaagttttcaagtacaaccggaagcctgcagagaccaatttaaggcactcgtgtaaacggataatggacatggtgagcaaccagcacccctggtttggaatggaacaggagtatactctgatgggaacagatgggcacccttttggttggccttccaatggctttcctgggccccaaggtccgtattactgtggtgtgggcgcagacaaagcctatggcagggatatcgtggaggctcactaccgcgcctgcttgtatgctggggtcaagattacaggaacaaatgctgaggtcatgcctgcccagtgggagttccaaataggaccctgtgaaggaatccgcatgggagatcatctctgggtggcccgtttcatcttgcatcgagtatgtgaagactttggggtaatagcaacctttgaccccaagcccattcctgggaactggaatggtgcaggctgccataccaactttagcaccaaggccatgcgggaggagaatggtctgaagtaagtagcttcctctggagccatctttattctcatggggtggaagggctttgtgttagggttgggaaagttggacttctcacaaactacatgccatgctcttcgtgtttgtcataagcctatcgttttgtacccgttggagaagtgacagtactctaggaatagaattacagctgtgatatgggaaagttgtcacgtaggttcaagcatttaaaggtctttagtaagaactaaatacacatacaagcaagtgggtgacttaattcttactgatgggaagaggccagtgatgggggtcttcccatccaaaagataattggtattacatgttgaggactggtctgaagcacttgagacataggtcacaaggcagacacagcctgcatcaagtatttattggtttcttatggaactcatgcctgctcctgcccttgaaggacaggtttctagtgacaaggtcagaccctcacctttactgcttccaccaggcacatcgaggaggccatcgagaaactaagcaagcggcaccggtaccacattcgagcctacgatcccaaggggggcctggacaatgcccgtcgtctgactgggttccacgaaacgtccaacatcaacgacttttctgctggtgtcgccaatcgcagtgccagcatccgcattccccggactgtcggccaggagaagaaaggttactttgaagaccgccgcccctctgccaattgtgacccctttgcagtgacagaagccatcgtccgcacatgccttctcaatgagactggcgacgagcccttccaatacaaaaactaattagactttgagtgatcttgagcctttcctagttcatcccaccccgccccagctgtctcattgtaactcaaaggatggaatatcaaggtctttttattcctcgtgcccagttaatcttgcttttattggtcagaatagaggagtcaagttcttaatccctatacacccaaccctcatttcttttctatttagctttctagtgggggtgggaggggtaggggaagggaacgtaaccactgcttcatctcatcaggaatgcatgtccagtaggcagagctgccacagagtgggtgtatttgtggaggaggactttttcttcaggacagttaaaagagcaggtccactgcttggattgacaattcccctataggtagagagctgctagttcttcaggtaaaaccaactttctattccaaatggaagttaggtgaggagtagtgggaggagttcatgccctccatgaagacagctcagtgtatcacctgacagatgggtagccctactgtaaaagaaggaaaagttatttctgggtcctccatttataacacaaagcagagtagtatttttatatttaaatgtaaaaacaaaagttatatatatggatatgtggatatatgtgtatttctaattgaggaaaccatcctagttactgggtttgccaagtttgaagagcttggttaacaagaaaggatctcttgagtagaggtgggggtgcagtaccaggaaaggtggttatctggggctcagcgctttattactatgtggggtttccctgcccactctgcaggagcagatgctggacaggtagcagggtgggacaccagtgcttgccaccacctgtccctgtgcttaggctaagatgcatatgtatccacacagagttagcaggatggagttggctggtcaacttgaacattgttactgataggggtgggtggggtttattttttggtgggactagcatgtcactaaagcaggccttttgatatattaaattttttaaagcaaaacaagttcagcttttaatcaactttgtagggtttctaactttacagaattgcctgtttgtttcagtgtctccatccactttgctcttggaggaacggaggacaggcagacctggagttaaaacatttgtcattttgtgtcatagtgtctactttctcccagcagaatattcctttccttcttaggagtcctatggagttttgtttttgttttttttctattacgataaacataccccacctccattctggcttgccctgctgttctctggttgtttgtgtgctgtccgcagcaggctgcctgtggttttctcttgccatgacgacttctaattgccatgtacagtatgttcagttagataactcctcattgtaaacagactgtaactgccagagcagcgcttataaatcaacctaacatttataagatttcctcttgacttgtttctttgtggttgggggaggaagaaaaaaaaaagcgtgcagtatttttttgttccttcatttcctatcaaaagaaaggggagtggttctgttttgtttactcgcaaaataagctagcttatctattggcttttctttttttttttttttttaaacgggctttttcttgtacctataatttggggtaaggtgtgagagtttttatagttttttgagacagggtcttggtgtatacccttggctggcctggagctaactatgtagactgggctagcctttaacttgcagttctgctttcaattagggtttatacatttagtcttggcaattcctagttccacgtttaatctctttacatttcaaagcagtgttatctgaagagttcaggcgcagagtcaattcaatagagttacacaaaaacctaaaaaacaagttttaaataccaagttatgttggcctggccacttttcacagctgtccacaactcaatgtgacaaggctacaaattggatatactagaatttcctggtgatttggaacccctgcttcatttcccggaaccagggcttttggtgacagtcctagcttatcagattatttaaaacagttactcttcctgcccttcttcctgagacctttgtccagctgccatgagccatctacacagtacttgcttccctgttgaagtcactgaaggcacatcagcccaagacataaaggcttgtcccggattcactagcctggtgaacttgtggttctctgatgttttgtcctgttttgttgtgatttagtctcaaatttcccagcctggtttgaaaatctgggctcccagccttcaataaggaggactacagatatgtacgactgagccttgattccagcctcatgtttatacgtctgtgctcagctccctgaaggttccagtttgaaactcaataatccaggggtcagaaagtcttgatcttatccccacagtatggcaccaagcctggctgagccttctgacttagtctgccctgttgctatttaagcacttttcttcactaggctaaaaataaaaggagcttcctcctttgccatggcgctgtgcatgataggaaaaggtagctatctactagcatattaactccactgtttttgctttgtgtgtttggtttttgaggaagggtctcaactgtgtatccctggctggcctggccggatctagcttcgtgtcaaggacggtgactgcagtgaataataaaatgtgtgtttgtccgaaatacgcgttttgagatttctgtcgccgactaaattcatgtcgcgcgatagtggtgtttatcgccgatagagatggcgatattggaaaaatcgatatttgaaaatatggcatattgaaaatgtcgccgatgtgagtttctgtgtaactgatatcgccatttccccaaaagtgatttttgggcatacgcgatatctggcggatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacgaagcttgccgccaccatgccgctgctgctactgctgcccctgctgtgggcaggggccctggctatggatcatcaccatcaccatcaccatcacggtggcggtctgaacgacatcttcgaggctcagaaaatcgaatggcacgaacggaaaatccccaaagtaggacatacttttttccaaaagcctgagagttgcccgcctgtgccaggaggtagtatgaaactcgacattggcatcatcaatgaaaaccagcgcgtttccatgtcacgtaacatcgagagccgctccacctccccctggaattacactgtcacttgggaccccaaccggtacccctcggaagttgtacaggcccagtgtaggaacttgggctgcatcaatgctcaaggaaaggaagacatctccatgaattccgttcccatccagcaagagaccctggtcgtccggaggaagcaccaaggctgctctgtttctttccagttggagaaggtgctggtgactgttggctgcacctgcgtcaccccagtcatccaccatgtgcagtaatgactcgagcaattggctagagtcgacgcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagcaaaatcactagt

SEQ ID NO: 22 - DNA, 321 bp, Mus musculus
gacattgtgatgacccagtctccagccaccctgtctgtgactccaggtgatagagtctctctttcctgcagggccagccagagtatcagcgaccacttacactggtatcaacaaaaatcacatgagtctccacggcttctcatcaaatatgcttcccatgccatttctgggatcccctccaggttcagtggcagtggatcagggacagatttcactctcagcatcaaaagtgtggaacctgaagatattggggtgtattactgtcaaaatggtcacagttttccgctcacgttcggtgctgggaccaagctggagctgaaa

SEQ ID NO: 23 - DNA, 354 bp, Mus musculus
gatgtgcagcttcaggagtcaggacctgacctaatacaaccttctcagtcactttcactcacctgcactgtcactggctactccatcaccggtggttatagctggcactggatccggcagtttccaggaaacaaactggaatggatgggctacatccactacagtggttacactgacttcaacccctctctcaaaactcgaatctctatcactcgagacacatccaagaaccagttcttcctgcagttgaattctgtgactactgaagacacagccacatattactgtgcaagaaaagatccgtccgacggatttccttactggggccaagggactctggtcactgtctctgca

(19) United States
(12) Patent Application Publication
(10) Pub. No.: US 2010/0093087 A1
(43) Pub. Date: Apr. 15, 2010

(54) IL-17 Mediated Transfection Methods

(75) Inventors: Greg Elson, Collonges sous Saleve (FR); Mathias Contie, Annemasse (FR); Nicolas Fouque, Collonges sous Saleve (FR); Olivier Leger, St.-Sixt (FR); Yves Poitevin, Ambilly (FR)

Correspondence Address: MINTZ, LEVIN, COHN, FERRIS, GLOVSKY AND POPEO, P.C, ONE FINANCIAL CENTER, BOSTON, MA, 02111, US

(73) Assignee: Novlmmune S.A., Geneva (CH)

(21) Appl. No.: 12/575,258

(22) Filed: Oct. 7, 2009

Related U.S. Application Data
(60) Provisional application No. 61/195,436, filed on Oct. 7, 2008.

(51) Int. Cl. C12N 5/00

(52) U.S. Cl. 435/375

(57) ABSTRACT

The invention comprises compositions and methods for IL-17-mediated transfection that results in superior and enhanced properties of cell survival and protein production.

### RELATED APPLICATIONS

This application claims the benefit of U.S. Provisional Application No. 61/195,436, filed Oct. 7, 2008, the contents of which are hereby incorporated by reference in their entirety.

### FIELD OF THE INVENTION

This invention relates generally to the fields of cell biology, cell culture and molecular biology. The invention comprises compositions and methods for using interleukin 17 (IL-17) and related proteins to produce superior and enhanced properties of gene delivery, cell survival, colony outgrowth and protein production.

### BACKGROUND OF THE INVENTION

In the fields of cell biology, cell culture and molecular biology, it is desirable to select cell lines having particular characteristics such as, for example, speed of growth, number of clones produced, productivity. Multiple methods for producing and selecting cell lines have been developed; however, there is an ongoing need for improving the efficiency, selection and other properties of cell lines.

### SUMMARY OF THE INVENTION

The invention provides compositions and methods for using an IL-17 composition to enhance a property of a cell line, to enhance subcloning of a cell, cell line or population, to enhance selection of a cell line, and/or to enhance expression of one or more exogenous gene(s) within selected cell lines. The methods and compositions encompassed by the invention represent a novel method of using IL-17 to enhance one or more characteristics and/or biological effects of a cell and/or a cell line. These IL-17-mediated methods and compositions are useful in producing, subcloning and/or selecting cells and/or cell lines that exhibit one or more desirable properties, characteristics or other biological effects. Moreover, when IL-17 is used in combination with known methods, one or more properties of cell line expression, selection, subcloning and/or efficacy are unexpectedly successful. For example, the encompassed compositions and methods of the invention provide a greater yield of monoclonal antibodies to be used in pharmaceutical compositions to be administered to patients in need thereof. Moreover, the instant methods allow the use of formerly transfection-resistant cell lines in research for the development of therapeutic compositions. Finally, the instant methods allow for fast, efficient, screening of selected cells on a large scale because the use of IL-17 increases the efficiency, productivity and/or speed of cell selection, subcloning and/or single cell cloning, exogenous gene expression and other desirable characteristics. Thus, the methods provided by the invention are applicable for large-scale drug development. Compositions and methods provided herein are also used in cell and tissue culture supplements and derivatives.

The methods and compositions provided herein enhance one or more properties of a cell and/or cell line, cell selection, subcloning, and/or of cell modification, including, for example, cell transfection. Exemplary properties which are enhanced by the use of IL-17 include, but are not limited to, increased efficiency, increased selection rate, increased cell growth, increased appearance speed of selected cells (i.e., the time it takes for the first appearance of the selected cells), increased number of selected cell lines, increased doubling time of selected cells, increased cell viability, reduced sensitivity to medium depletion, and/or increased cell line stability. In some embodiments, the methods and compositions provided herein enhance any combination of two or more of the properties described above.

Specifically, the invention provides a method of using IL-17 to enhance a property of cell and/or cell line production, cell and/or cell line selection, subcloning, and/or cell and/or cell line transfection with a nucleic acid, the method including the step of contacting the cell with the IL-17. Preferably, the exposure to exogenous IL-17 causes enhanced cell production, selection, subcloning, and/or expression of the nucleic acid compared to a cell not contacted by IL-17. The exogenous IL-17 is, for example, from cells that have been transformed to express IL-17.

The invention provides compositions and methods of using IL-17 to enhance the efficacy of cell production, subcloning, single cell cloning, and/or selection, including the steps of: culturing one or more cells or cell line(s) in medium and contacting the cell(s) and/or cell line(s) with an IL-17 containing composition to enhance a property of the cell and/or cell line such as, for example, increased efficiency, increased selection rate, increased cell growth, increased appearance speed of selected cells (i.e., the time it takes for the first appearance of the selected cells), increased number of selected cell lines, increased doubling time of selected cells, increased cell viability, reduced sensitivity to medium depletion, and/or increased cell line stability. Optionally, the cell(s) and/or cell line(s) are contacted with a nucleic acid and cultured in medium to express a polypeptide encoded by the nucleic acid such that one or more cells and/or cell lines expressing one or more polypeptides is generated, wherein the generated cell(s) and/or cell line(s) demonstrate an enhanced property of transfection. The cell(s) and/or cell line(s) are exposed to IL-17 prior to or during the time the cell(s) and/or cell line(s) are contacted with the nucleic acid encoding the polypeptide of interest.

The invention further provides a method of enhancing the efficacy of cell modification, including the steps of: (a) culturing one or more cells or cell line(s) in medium; (b) contacting one or more cells or cell line(s) with a nucleic acid; (c) culturing modified cells in medium to express the polypeptide encoded by the nucleic acid wherein cells are exposed to IL-17 prior to or during the contacting step; and wherein one or more cell lines expressing one or more polypeptides is generated that demonstrates an enhanced property of transfection.

The invention further provides a method of enhancing the efficacy and/or productivity of subcloning and/or single cell cloning in which one or more transformed cell(s) or cell line(s) are cultured in medium and contacted with, or otherwise exposed to IL-17, wherein the contacted cell(s) or cell line(s) demonstrates an enhanced property of subcloning and/or single cell cloning. The compositions and methods are used to enhance a property of subcloning and/or single cell cloning, such as, for example, increased efficiency, increased selection rate, increased cell growth, increased appearance speed of selected cells (i.e., the time it takes for the first appearance of the selected cells), increased number of selected cell lines, increased doubling time of selected cells, increased cell viability, reduced sensitivity to medium depletion, and/or increased cell line stability. For example, the method is used to enhance the efficacy, efficiency, productivity and/or selection of subcloning and/or single cell cloning of one or more eukaryotic, e.g., human cell(s). In some embodiments, the cell(s) are cultured in serum-free medium, preferably in chemically defined medium. The methods provided herein are useful in subcloning eukaryotic cell lines even at very low cell line densities, such as, for example, in the range of 1 cell/mL to 10,000 cells/mL, in the range of 1 cell/mL to 5,000 cells/mL, in the range of 1 cell/mL to 500 cells/mL, in the range of 1 cell/mL to 250 cells/mL, in the range of 1 cell/mL to 100 cells/mL, in the range of 1 cell/mL to 50 cells/mL, in the range of 1 cell/mL to 25 cells/mL, in the range of 1 cell/mL to 12.5 cells/mL, in the range of 1 cell/mL to 6.25 cells/mL, or in the range of 1 cell/mL to 3.125 cells/mL.

In one embodiment, the cells and/or cell lines are transfected with a first nucleic acid encoding an IL-17 cytokine, preferably IL-17F, and a second nucleic acid encoding a peptide, polypeptide, or protein of interest, and the cells are cultured under conditions suitable for the expression of the first and second nucleic acids. Alternatively, the first nucleic acid encoding an IL-17 cytokine, preferably IL-17F, and the second nucleic acid encoding a peptide, polypeptide, or protein of interest are transfected into two different cells and/or cell lines, and the cells are cultured together under conditions suitable for the expression the first and second nucleic acids. In these IL-17 transfected cells and/or cell lines, the co-expression of the IL-17 cytokine along with the peptide, polypeptide or protein of interest causes an increase in one or more transfection properties, such as, for example, increased efficiency, increased selection rate, increased cell growth, increased appearance speed of selected cells, increased number of selected cell lines, increased doubling time of selected cells, increased cell viability, reduced sensitivity to medium depletion, and/or increased cell line stability.

In a more preferred embodiment, the cells and/or cell lines are transfected with a first nucleic acid encoding an IL-17 cytokine, preferably IL-17F, and a second nucleic acid encoding a peptide, polypeptide, or protein of interest, wherein the expression of the IL-17-encoding nucleic acid is regulated by any of a variety of art-recognized methods, including, for example, the use of an inducible promoter, inactivation by CreLoxP or an equivalent, or zinc finger inactivation downstream of the selection and/or subcloning process. Alternatively, the first nucleic acid encoding an IL-17 cytokine, preferably IL-17F, and the second nucleic acid encoding a peptide, polypeptide, or protein of interest are transfected into two different cells and/or cell lines, and the cells are cultured together under conditions suitable for the expression the first and second nucleic acids. The cells and/or cell lines are then cultured under conditions suitable for the expression of the first and second nucleic acids.

In these IL-17 transfected cells and/or cell lines, the regulated, co-expression of the IL-17 cytokine along with the peptide, polypeptide or protein of interest causes an increase in one or more transfection properties, such as, for example, increased efficiency, increased selection rate, increased cell growth, increased appearance speed of selected cells, increased number of selected cell lines, increased doubling time of selected cells, increased cell viability, reduced sensitivity to medium depletion, and/or increased cell line stability.

In the compositions and methods provided herein, IL-17 expression is regulated by any of a variety of art-recognized methods, including, for example, the use of an inducible promoter, inactivation by CreLoxP or an equivalent, or zinc finger inactivation downstream of the selection and/or subcloning process. Suitable inducible promoters include, for example, heterologous gene regulation systems such as systems that use rapamycin-inducing dimerizing technology, steroid-hormone receptor-based systems, tetracycline systems such as the TET system, streptogramin systems such as the —PIP system, and macrolide systems such as the E.EREX system. In these heterologous gene regulation systems, the regulatory sequence is fused to the partial sequence of a strong promoter such as the hCMV promoter or Ef1 alpha promoter.

IL-17 contacts a cell prior to, during, or following the cell selection and/or modification. Alternatively, or in addition, IL-17 contacts a cell continuously. Contemplated within the above methods are several means by which IL-17 contacts cells. In one embodiment, IL-17 contacts a cell by being present in the culture medium. In another embodiment, IL-17 is produced exogenously by a cell, for example, the IL-17 is produced by cell(s) that have been transformed to express IL-17. In a related embodiment, the nucleic acid of the above method comprises one or more sequences encoding an IL-17 cytokine. Moreover, IL-17 is produced simultaneously or sequentially with the nucleic acid.

The above methods encompass a cell or cell lines under selective pressure. In one embodiment, the selective pressure is applied by growing transfected cells in a medium comprising a specific glutamine synthetase inhibitor, wherein transfected cells survive, and untransfected cells die. In a preferred embodiment, the specific glutamine synthetase inhibitor is methionine sulphoximine (MSX). Increase of selection pressure on the cell selection, for example, by increasing the concentration of MSX in the medium (e.g., above 50 μM) in the presence of an IL-17 cytokine, preferably IL-17F, increased the productivity. Increasing selective pressure in the absence of an IL-17 cytokine, preferably IL-17F, resulted in the absence of clones. Thus, the addition of IL-17F and increasing the selective pressure increases the productivity of the methods provided herein.

When selective pressure is applied, the modification is semi-stable. Alternatively, when selective pressure is applied, the modification is stable. In another embodiment the modified cells are grown in the absence of selective pressure, and therefore, the modification is transient.

The methods and compositions use an IL-17 polypeptide, also referred to herein as an IL-17 cytokine, to enhance one or more properties of cell transfection. Exemplary IL-17 polypeptides, or cytokines encompassed by the invention include, but are not limited to, IL-17A, IL-17B, IL-17C, IL-17D, IL-17E, or IL-17F, along with heterodimers of these IL-17 polypeptides, such as, for example, the IL-17A/IL-17F heterodimer. In a preferred embodiment, an IL-17F cytokine is used. The IL-17 polypeptides are, for example, human IL-17 sequences, including the human IL-17 sequences shown herein. In some embodiments, the IL-17 polypeptides and IL-17 compositions include eukaryotic sequences including non-human, mammalian, sequences such as, for example, rat IL-17 sequences. In one embodiment, cells or cell lines(s) include Th17 cells which secrete an IL-17 polypeptide. In some embodiments, cells or cell line(s) of the above methods express at least one IL-17 receptor. Exemplary IL-17 receptors (IL-17Rs) include, but are not limited to, IL-17RA, IL-17RB, IL-17RC, IL-17RD, and IL-17RE.

Methods of the invention include cells that receive one or more DNA and/or IL-17 compositions and grow in culture under selective pressure to retain these compositions. In one embodiment, the selective pressure is applied by growing transfected cells in a medium comprising a specific glutamine synthase inhibitor, wherein transfected cells that receive the DNA composition survive, and untransfected cells die. In a preferred embodiment, the specific glutamine synthase inhibitor is methionine sulphoximine (MSX). In another embodiment, the DHFR (Dihydrofolate reductase)-deficient transfected cells are selected by using a culture medium deficient in hypoxanthine and thymidine (HT medium). In some embodiments, methotrexate (MTX) is used in the system for selection and gene amplification purposes.

Methods and compositions of the invention enhance a property of transfection. Methods and compositions of the invention enhance a property of cell production. Methods and compositions of the invention enhance a property of selection. Methods and compositions of the invention enhance a property of subcloning and/or single cell cloning. Exemplary properties which are enhanced by the instant methods include, but are not limited to, increased transfection efficiency, increased selection rate, increased transfected cell growth, increased appearance speed of selected cells, increased number of selected cell lines, increased doubling time of selected cells, increased cell viability, or increased cell line stability.

Methods of the invention enhance expression of one or more exogenous gene(s). Exemplary mechanisms by which expression is enhanced include, but are not limited to, increased specific production rate of monoclonal antibody (MAb), increased MAb titer, increased product quality, correlation of IL-17 expression with MAb titer, increased expression following transient transfection of transfection-resistant cell-lines, or increased transgene productivity, increased incorporation of exogenous DNA into genomic sequence, increased retention of exogenous DNA, increased uptake of DNA, or increased expression of exogenous DNA.

The invention provides a method of enhancing the selection rate of semi-stable transfection, including the steps of: (a) culturing a serum-free suspension-adapted Chinese Hamster Ovary (CHO) cell line in glutamine-depleted medium; (b) mixing the CHO cell line with a DNA composition including sequences encoding for a human IL-17F and a glutamine synthase gene; (c) transporting one or more DNA compositions across the plasma membranes of at least one cell line by electroporation; (d) culturing transfected cells in the glutamine-depleted medium under selective pressure by adding MSX, e.g., in a concentration of 50 μM MSX or 100 μM MSX, at a concentration in a range from 50 μM MSX to 100 μM MSX, or at a concentration greater than 100 μM MSX to the medium; and (e) allowing transfected cells to express polypeptides encoded by the transfected DNA compositions under selective pressure; wherein a mixture of cell lines expressing one or more polypeptides is generated that demonstrates an enhanced property of transfection.

The invention further provides a method of enhancing the selection rate of stable transfection, including the steps of: (a) culturing a serum-free suspension-adapted Chinese Hamster Ovary (CHO) cell line in glutamine-depleted medium; (b) mixing the CHO cell line with a DNA composition including sequences encoding for a human IL-17F and a glutamine synthase gene; (c) transporting one or more DNA compositions across the plasma membranes of at least one cell line by electroporation; (d) culturing transfected cells in the glutamine-depleted medium under selective pressure by adding MSX, e.g., in a concentration of 50 μM MSX or 100 μM MSX, at a concentration in a range from 50 μM MSX to 100 μM MSX, or at a concentration greater than 100 μM MSX to the medium; and (e) allowing transfected cells to express polypeptides encoded by the transfected DNA compositions under selective pressure; wherein an isolated cell line expressing one or more polypeptides is generated that demonstrates an enhanced property of transfection.

The invention encompasses a method of enhancing the selected cell numbers of semi-stable transfection, including the steps of: (a) culturing a serum-free suspension-adapted Chinese Hamster Ovary (CHO) cell line in glutamine-depleted medium; (b) mixing the CHO cell line with a DNA composition comprising sequences encoding for a human IL-17F and a glutamine synthase gene; (c) transporting one or more DNA compositions across the plasma membranes of at least one cell line by electroporation; (d) culturing transfected cells in the glutamine-depleted medium under selective pressure by adding MSX, e.g., in a concentration of 50 μM MSX or 100 μM MSX, at a concentration in a range from 50 μM MSX to 100 μM MSX, or at a concentration greater than 100 μM MSX to the medium; and (e) allowing transfected cells to express polypeptides encoded by the transfected DNA compositions under selective pressure; wherein a mixture of cell lines expressing one or more polypeptides is generated that demonstrates an enhanced property of transfection.

The invention further encompasses a method of enhancing the selected cell numbers of stable transfection, comprising the steps of: (a) culturing a serum-free suspension-adapted Chinese Hamster Ovary (CHO) cell line in glutamine-depleted medium; (b) mixing the CHO cell line with a DNA composition comprising sequences encoding for a human IL-17F and a glutamine synthase gene; (c) transporting one or more DNA compositions across the plasma membranes of at least one cell line by electroporation; (d) culturing transfected cells in the glutamine-depleted medium under selective pressure by adding MSX hi a concentration of 50 μM MSX or 100 μM MSX, at a concentration in a range from 50 μM MSX to 100 μM MSX, or at a concentration greater than 100 μM MSX to the medium; and (e) allowing transfected cells to express polypeptides encoded by the transfected DNA compositions under selective pressure; wherein an isolated cell line expressing one or more polypeptides is generated that demonstrates an enhanced property of transfection.

### BRIEF DESCRIPTION OF THE DRAWINGS

FIG. 1 is a graph comparing stable transfection between human IL-17F and an anti-RANTES monoclonal antibody (referred to herein as NI-0701, described in PCT Publication No. WO 09/054,873) for speed and rate of colony emergence. Error bars represent standard deviation of 2 independent experiments.

FIG. 2A is a graph comparing stable transfections using human IL-17F for presence of multiple transfectants per well.

FIG. 2B is a graph comparing stable transfections using NI-0701 for presence of multiple transfectants per well.

FIG. 3 is a series of photographs illustrating the visual examination of semi-stable transfection pools expressing human IL-17F. Pictures were taken with the aid of a fluorescence microscope under 100× magnification at the indicated time points.

FIG. 4A is a graph comparing semi-stable transfections of the A6VL construct either supplemented or not with recombinant Human IL-17F for GFP expression.

FIG. 4B is a graph comparing semi-stable transfections of the A6VL construct either supplemented or not with recombinant Human IL-17F for cell viability.

FIG. 5 is a graph comparing the stable transfection between human IL-17F, human IL-17A and A6VL constructs for speed and rate of colony emergence. Error bars represent standard deviation of 2 independent experiments.

FIG. 6A is a graph comparing the stable transfection between human IL-17F, rat IL-17F and A6VL constructs. Stable transfections were assessed for speed and rate of colony emergence. Error bars represent standard deviation of 2 independent experiments.

FIG. 6B is a graph comparing the semi-stable transfection between human IL-17F, rat IL-17F and A6VL constructs. Semi-stable transfections were assessed for GFP expression. Error bars represent standard deviation of 2 independent experiments.

FIG. 6C is a graph comparing the semi-stable transfection between human IL-17F, rat IL-17F and A6VL constructs. Semi-stable transfections were assessed for cell viability. Error bars represent standard deviation of 2 independent experiments.

FIG. 7A is a graph comparing the stable transfection between human IL-17F, and A6VL constructs in CHO—S cell line (Invitrogen), which were assessed for speed and rate of colony emergence.

FIG. 7B is a graph comparing the semi-stable transfection between human IL-17F, and A6VL constructs in CHO—S cell line, which were assessed for GFP expression.

FIG. 7C is a graph comparing the semi-stable transfection between human IL-17F, and A6VL constructs in CHO—S cell line, which were assessed for cell viability.

FIG. 8A is a graph comparing the stable transfection of IL-17 IRES GFP variants into CHO cells using an expression vector system based on puromycin selection (pEAK8, Edge Biosystems). GFP expression analysis was measured using flow cytometry 24 hours post-transfection in PEAK cells.

FIG. 8B is a graph comparing the GFP-expression in CHO cells after 3 weeks of selection with puromycin following the transfection procedure described in the description of FIG. 8A.

FIG. 9A is a graph comparing the production of an anti-CD3 monoclonal antibody (referred to herein as the 15C1 MAb and described in PCT Publication No. WO 05/118635) (μg/mL) from 1 to 4 weeks following transfection of CHO cells with either a combination of the IL-17F expression vector and the 15C1 MAb Double Gene Expression Vector or the 15C1 MAb Double Gene Expression Vector alone.

FIG. 9B is a graph comparing the number of wells containing 1 or more colonies per 96 well-plate at 22 and 26 days following transfection of CHO cells with either a combination of the IL-17F expression vector and the 15C1 MAb Double Gene Expression Vector or the 15C1 MAb Double Gene Expression Vector alone.

FIG. 9C is a graph comparing the level of expression of 15C1 MAb (μg/mL) in the supernatant of each of 20 clones following transfection of CHO cells with either a combination of the IL-17F expression vector and the 15C1 MAb Double Gene Expression Vector or the 15C1 MAb Double Gene Expression Vector alone.

FIG. 10 is a schematic representation, or map, of the pEE14.4 LSCD33HIS AVI hIL-17F n 1-7 Expression Vector.

FIG. 11A is a graph depicting the quantification of isolated clones picked three days after plating cells from two CHOK1SV cell lines, 8E11, which expresses IL-17F-IRES-GFP, C6C5, which expresses an irrelevant mAb.

FIGS. 11B and 11C are illustrations depicting the subclones picked in FIG. 11A.

FIG. 12 is a graph depicting the GFP expression in clones from cells transfected with an IL-17F-IRES-GFP-expression cassette and plated under 50 μM or 100 μM MSX selection pressure.

FIG. 13 is a series of illustrations depicting vector constructs used in the examples provided herein.

FIG. 14 is a graph depicting the appearance of stable CHODG44 cell clones at various times post-transfection.

FIG. 15 is a graph depicting the level of clonal GFP expression in CHODG44 cells after 5 weeks of selection under MTX pressure.

FIG. 16 is a graph depicting graph depicting the appearance of stable CHO cell clones at various times post-transfection

FIG. 17 is an illustration depicting the average level of IgG expression of individual clones at four weeks post-transfection.

### DETAILED DESCRIPTION

The invention provides compositions and methods for using an IL-17 composition to enhance a property of transfection and to enhance expression of one or more exogenous gene(s) within transfected cell lines. The methods encompassed by the invention represent a novel method of transfection mediated by IL-17. Moreover, when IL-17 is used in combination with known methods, one or more properties of transfection efficacy, e.g., survival, growth and/or transgene expression, are unexpectedly successful.

### IL-17 Compositions

IL-17 compositions include one or more polynucleotide sequences encoding for an IL-17 cytokine. Encompassed IL-17 cytokines include, but are not limited to, IL-17A, IL-17B, IL-17C, IL-17D, IL-17E, and IL-17F (isoforms 1 and 2, also known as ML-1). Preferred IL-17 cytokines are the two isoforms of IL-17F. IL-17 compositions include one or more polypeptide sequences comprising an IL-17 cytokine. Furthermore, IL-17 compositions include one or more polynucleotide or polypeptide sequences containing an IL-17 cytokine receptor (IL-17R). Encompassed IL-17 cytokine receptors include, but are not limited to, IL-17RA, IL-17RB, IL-17RC, IL-17RD, and IL-17RE. IL-17 compositions also include polypeptides and proteins that have similar structures to one or more of the IL-17 cytokines and/or IL-17R receptors described herein. IL-17 compositions also include fragments or other processed portions of one or more of the IL-17 cytokines and/or IL-17R receptors described herein, for example, fragments that are derived from intracellular processing of the IL-17 cytokine, IL-17R receptor and any homodimer or heterodimer thereof. In one embodiment of the invention, compositions including at least one IL-17 cytokine are administered to a cell or cell lines which express, overexpress, or repress expression of at least one IL-17R. In this embodiment, the dosage of IL-17 cytokine present in the composition is modified, either increased or decreased to compensate for the expression level of the IL-17R. For instance, when expression levels of the IL-17R are high, the composition includes lower levels of at least one IL-17 cytokine. Conversely, when expression of at least one IL-17R is low, compositions include higher levels of at least one IL-17 cytokine.

Encompassed human IL-17 sequences are shown below, however, IL-17 compositions include eukaryotic sequences including non-human, mammalian, sequences. IL-17 compositions further include one or more mutations at any point along these sequences. Contemplated mutations disrupt one or more functions of an IL-17 cytokine. For example, a contemplated mutation prevents IL-17 binding to or releasing from an IL-17 receptor. Alternatively, or in addition, a contemplated mutation prevents IL-17 expression, translation, secretion, dimerization, or degradation. IL-17 mutations cause IL-17 aggregate extracellularly or intracellularly. Mutations at the polynucleotide level are silent or, alternatively, cause changes in the polynucleotide or amino acid sequence, including reading frame shifts, substitutions, deletions, inversions, missense mutations, or terminations. Mutations at the polypeptide level are silent or, alternatively, cause changes in the amino acid sequence, prevention or termination of translation, disruption of tertiary structure, misfolding, aggregation, disruption of dimerization, disruption of degradation, protein instability, disruption of interactions with other polypeptides or novel associations with polypeptides.

In a preferred embodiment, the IL-17 composition includes the human cytokine interleukin 17F (IL-17F or hIL-17F) isolated from human cDNA or the rat cytokine interleukin 17F (rIL-17F) and subsequently sub-cloned into an expression vector under the control of the hCMV promoter. In this expression vector, GFP is cloned downstream of the hIL-17F cDNA as a second cistron under the control of the same CMV promoter. The two cistrons (IL-17F and GFP) are separated by a viral internal ribosome entry site (IRES) to allow for translation of the second (GFP) cistron. The vector also contains the glutamine synthase (GS) gene under the control of the SV40 promoter for selection of transfected cells in glutamine-free medium using MSX.

In some embodiments, the vectors described herein also include a tag or other marker (or a nucleic acid sequence encoding for the tag or marker) such as, for example, an Avi-tag, a His tag. In other embodiments, the vectors do not contain a tag or nucleic acid sequence encoding a tag.

Contemplated human IL-17 cytokines are described, for example, but not limited by, the following sequences. Mutations are engineered at one or more positions along the mRNA or amino acid sequences of the following:

IL-17A is encoded by the following mRNA sequence (NCBI Accession No. NM_{—}002190 and SEQ ID NO: 1):

| | |
|---|---|
| 1 | gcaggcacaa actcatccat ccccagttga ttggaagaaa caacgatgac tcctgggaag |
| 61 | acctcattgg tgtcactgct actgctgctg agcctggagg ccatagtgaa ggcaggaatc |
| 121 | acaatcccac gaaatccagg atgcccaaat tctgaggaca agaacttccc ccggactgtg |
| 181 | atggtcaacc tgaacatcca taaccggaat accaatacca atcccaaaag gtcctcagat |
| 241 | tactacaacc gatccacctc accttggaat ctccaccgca atgaggaccc tgagagatat |
| 301 | ccctctgtga tctgggaggc aaagtgccgc cacttgggct gcatcaacgc tgatgggaac |
| 361 | gtggactacc acatgaactc tgtccccatc cagcaagaga tcctggtcct gcgcagggag |
| 421 | cctacacact gccccaactc cttccggctg gagaagatac tggtgtccgt gggctgcacc |
| 481 | tgtgtcaccc cgattgtcca ccatgtggcc taagagctct ggggagccca cactccccaa |
| 541 | agcagttaga ctatggagag ccgacccagc ccctcaggaa ccctcatcct tcaaagacag |
| 601 | cctcatttcg gactaaactc attagagttc ttaaggcagt ttgtccaatt aaagcttcag |
| 661 | aggtaacact tggccaagat atgagatctg aattaccttt ccctctttcc aagaaggaag |
| 721 | gtttgactga gtaccaattt gcttcttgtt tactttttta agggctttaa gttatttatg |
| 781 | tatttaatat gccctgagat aactttgggg tataagattc cattttaatg aattacctac |
| 841 | tttattttgt ttgtcttttt aaagaagata agattctggg cttgggaatt ttattattta |
| 901 | aaaggtaaaa cctgtattta tttgagctat ttaaggatct atttatgttt aagtatttag |
| 961 | aaaaaggtga aaaagcacta ttatcagttc tgcctaggta aatgtaagat agaattaaat |
| 1021 | ggcagtgcaa aatttctgag tctttacaac atacggatat agtatttcct cctctttgtt |
| 1081 | tttaaaagtt ataacatggc tgaaaagaaa gattaaacct actttcatat gtattaattt |
| 1141 | aaattttgca atttgttgag gttttacaag agatacagca agtctaactc tctgttccat |
| 1201 | taaaccctta taataaaatc cttctgtaat aataaagttt caaaagaaaa tgtttatttg |
| 1261 | ttctcattaa atgtatttta gcaaactcag ctcttcccta ttgggaagag ttatgcaaat |
| 1321 | tctcctataa gcaaaacaaa gcatgtcttt gagtaacaat gacctggaaa tacccaaaat |
| 1381 | tccaagttct cgatttcaca tgccttcaag actgaacacc gactaaggtt ttcatactat |
| 1441 | tagccaatgc tgtagacaga agcattttga taggaataga gcaaataaga taatggccct |
| 1501 | gaggaatggc atgtcattat taaagatcat atggggaaaa tgaaaccctc cccaaaatac |
| 1561 | aagaagttct gggaggagac attgtcttca gactacaatg tccagtttct cccctagact |
| 1621 | caggcttcct ttggagatta aggcccctca gagatcaaca gaccaacatt tttctcttcc |
| 1681 | tcaagcaaca ctcctagggc ctggcttctg tctgatcaag gcaccacaca acccagaaag |
| 1741 | gagctgatgg ggcagaacga actttaagta tgagaaaagt tcagcccaag taaaataaaa |
| 1801 | actcaatcac attcaattcc agagtagttt caagtttcac atcgtaacca ttttcgccc |

IL-17A is encoded by the following amino acid sequence (NCBI Accession No. NP_{—}002181.1 and SEQ ID NO: 2):

MTPGKTSLVSLLLLLSLEAIVKAGITIPRNPGCPNSEDKNFPRTVMVNLN
IHNRNTNTNPKRSSDYYNRSTSPWNLHRNEDPERYPSVIWEAKCRHLGCI
NADGNVDYHMNSVPIQQEILVLRREPPHCPNSFRLEKILVSVGCTCVTPI
VHHVA

IL-17B is encoded by the following mRNA sequence (NCBI Accession No. AF152098 and SEQ ID NO: 3):

| | |
|---|---|
| 1 | aggcgggcag cagctgcagg ctgaccttgc agcttggcgg aatggactgg cctcacaacc |
| 61 | tgctgtttct tcttaccatt tccatcttcc tggggctggg ccagcccagg agccccaaaa |
| 121 | gcaagaggaa ggggcaaggg cggcctgggc ccctggcccc tggccctcac caggtgccac |
| 181 | tggacctggt gtcacggatg aaaccgtatg cccgcatgga ggagtatgag aggaacatcg |
| 241 | aggagatggt ggcccagctg aggaacagct cagagctggc ccagagaaag tgtgaggtca |
| 301 | acttgcagct gtggatgtcc aacaagagga gcctgtctcc ctggggctac agcatcaacc |
| 361 | acgaccccag ccgtatcccc gtggacctgc cggaggcacg gtgcctgtgt ctgggctgtg |
| 421 | tgaacccctt caccatgcag gaggaccgca gcatggtgag cgtgccggtg ttcagccagg |
| 481 | ttcctgtgcg ccgccgcctc tgcccgccac cgccccgcac agggccttgc cgccagcgcg |
| 541 | cagtcatgga gaccatcgct gtgggctgca cctgcatctt ctgaatcacc tggcccagaa |
| 601 | gccaggccag cagcccgaga ccatcctcct tgcacctttg tgccaagaaa ggcctatgaa |
| 661 | aagtaaacac tgacttttga aagcaag |

IL-17B is encoded by the following amino acid sequence (NCBI Accession No. AAF28104.1 and SEQ ID NO: 4):

MDWPHNLLFLLTISIFLGLGQPRSPKSKRKGQGRPGPLAPGPHQVPLDLV
SRMKPYARMEEYERNIEEMVAQLRNSSELAQRKCEVNLQLWMSNKRSLSP
WGYSINHDPSRIPVDLPEARCLCLGCVNPFTMQEDRSMVSVPVFSQVPVR
RRLCPPPPRTGPCRQRAVMETIAVGCTCIF

IL-17C is encoded by the following mRNA sequence (NCBI Accession No. NM_{—}013278 and SEQ ID NO: 5):

| | |
|---|---|
| 1 | gccaggtgtg caggccgctc caagcccagc ctgccccgct gccgccacca tgacgctcct |
| 61 | ccccggcctc ctgtttctga cctggctgca cacatgcctg gcccaccatg acccctccct |
| 121 | cagggggcac ccccacagtc acggtacccc acactgctac tcggctgagg aactgcccct |
| 181 | cggccaggcc cccccacacc tgctggctcg aggtgccaag tgggggcagg ctttgcctgt |
| 241 | agccctggtg tccagcctgg aggcagcaag ccacaggggg aggcacgaga ggccctcagc |
| 301 | tacgacccag tgcccggtgc tgcggccgga ggaggtgttg gaggcagaca cccaccagcg |
| 361 | ctccatctca ccctggagat accgtgtgga cacggatgag gaccgctatc cacagaagct |
| 421 | ggccttcgcc gagtgcctgt gcagaggctg tatcgatgca cggacgggcc gcgagacagc |
| 481 | tgcgctcaac tccgtgcggc tgctccagag cctgctggtg ctgcgccgcc ggccctgctc |
| 541 | ccgcgacggc tcggggctcc ccacacctgg ggcctttgcc ttccacaccg agttcatcca |
| 601 | cgtccccgtc ggctgcacct gcgtgctgcc ccgttcagtg tgaccgccga ggccgtgggg |
| 661 | cccctagact ggacacgtgt gctccccaga gggcaccccc tatttatgtg tatttattgt |
| 721 | tatttatatg cctcccccaa cactaccctt ggggtctggg cattccccgt gtctggagga |
| 781 | cagcccccca ctgttctcct catctccagc ctcagtagtt gggggtagaa ggagctcagc |
| 841 | acctcttcca gcccttaaag ctgcagaaaa ggtgtcacac ggctgcctgt accttggctc |
| 901 | cctgtcctgc tcccggcttc ccttacccta tcactggcct caggcccccg caggctgcct |
| 961 | cttcccaacc tccttggaag tacccctgtt tcttaaacaa ttatttaagt gtacgtgtat |
| 1021 | tattaaactg atgaacacat ccccaaaa |

IL-17C is encoded by the following amino acid sequence (NCBI Accession No. NP_{—}037410.1 and SEQ ID NO: 6):

MTLLPGLLPLTWLHTCLAHHDPSLRGHPHSHGTPHCYSAEELPLGQAPPH
LLARGAKWGQALPVALVSSLEAASHRGRHERPSATTQCPVLRPEEVLEAD
THQRSISPWRYRVDTDEDRYPQKLAFAECLCRGCIDARTGRETAALNSVR
LLQSLLVLRRRPCSRDGSGLPTPGAFAFHTEFIHVPVGCTCVLPRSV

IL-17D is encoded by the following mRNA sequence (NCBI Accession No. NM_{—}138284 and SEQ ID NO: 7):

| | |
|---|---|
| 1 | aaaatgtttt cagctcctgg aggcgaaagg tgcagagtcg ctctgtgtcc gtgaggccgg |
| 61 | gcggcgacct cgctcagtcg gcttctcggt ccgagtcccc gggtctggat gctggtagcc |
| 121 | ggcttcctgc tggcgctgcc gccgagctgg gccgcgggcg ccccgagggc gggcaggcgc |
| 181 | cccgcgcggc cgcggggctg cgcggaccgg ccggaggagc tactggagca gctgtacggg |
| 241 | cgcctggcgg ccggcgtgct cagtgccttc caccacacgc tgcagctggg gccgcgtgag |
| 301 | caggcgcgca acgcgagctg cccggcaggg ggcaggcccg ccgaccgccg cttccggccg |
| 361 | cccaccaacc tgcgcagcgt gtcgccctgg gcctacagaa tctcctacga cccggcgagg |
| 421 | taccccaggt acctgcctga agcctactgc ctgtgccggg gctgcctgac cgggctgttc |
| 481 | ggcgaggagg acgtgcgctt ccgcagcgcc cctgtctaca tgcccaccgt cgtcctgcgc |
| 541 | cgcacccccg cctgcgccgg cggccgttcc gtctacaccg aggcctacgt caccatcccc |
| 601 | gtgggctgca cctgcgtccc cgagccggag aaggacgcag acagcatcaa ctccagcatc |
| 661 | gacaaacagg gcgccaagct cctgctgggc cccaacgacg cgcccgctgg cccctgaggc |
| 721 | cggtcctgcc ccgggaggtc tccccggccc gcatcccgag gcgcccaagc tggagccgcc |
| 781 | tggagggctc ggtcggcgac ctctgaagag agtgcaccga gcaaaccaag tgccggagca |
| 841 | ccagcgccgc ctttccatgg agactcgtaa gcagcttcat ctgacacggg catccctggc |
| 901 | ttgcttttag ctacaagcaa gcagcgtggc tggaagctga tgggaaacga cccggcacgg |
| 961 | gcatcctgtg tgcggcccgc atggagggtt tggaaaagtt cacggaggct ccctgaggag |
| 1021 | cctctcagat cggctgctgc gggtgcaggg cgtgactcac cgctgggtgc ttgccaaaga |
| 1081 | gatagggacg catatgcttt ttaaagcaat ctaaaaataa taataagtat agcgactata |
| 1141 | tacctacttt taaaatcaac tgttttgaat agaggcagag ctattttata ttatcaaatg |
| 1201 | agagctactc tgttacattt cttaacatat aaacatcgtt ttttacttct tctggtagaa |
| 1261 | ttttttaaag cataattgga atccttggat aaattttgta gctggtacac tctggcctgg |
| 1321 | gtctctgaat tcagcctgtc accgatggct gactgatgaa atggacacgt ctcatctgac |
| 1381 | ccactcttcc ttccactgaa ggtcttcacg ggcctccagg tggaccaaag ggatgcacag |
| 1441 | gcggctcgca tgccccaggg ccagctaaga gttccaaaga tctcagattt ggttttagtc |
| 1501 | atgaatacat aaacagtctc aaactcgcac aattttttcc cccttttgaa agccactggg |
| 1561 | gccaatttgt ggttaagagg tggtgagata agaagtggaa cgtgacatct ttgccagttg |
| 1621 | tcagaagaat ccaagcaggt attggcttag ttgtaagggc tttaggatca ggctgaatat |
| 1681 | gaggacaaag tgggccacgt tagcatctgc agagatcaat ctggaggctt ctgtttctgc |
| 1741 | attctgccac gagagctagg tccttgatct tttctttaga ttgaaagtct gtctctgaac |
| 1801 | acaattattt gtaaaagtta gtagttcttt tttaaatcat taaaagaggc ttgctgaagg |
| 1861 | aaaaaaaaaa aaa |

IL-17D is encoded by the following amino acid sequence (NCBI Accession No. NP_{—}612141.1 and SEQ ID NO: 8)

MLVAGFLLALPPSWAAGAPRAGRRPARPRGCADRPEELLEQLYGRLAAGV
LSAFHHTLQLGPREQARNASCPAGGRPADRRFRPPTNLRSVSPWAYRISY
DPARYPRYLPEAYCLCRGCLTGLFGEEDVRFRSAPVYMPTVVLRRTPACA
GGRSVYTEAYVTIPVGCTCVPEPEKDADSINSSIDKQGAKLLLGPNDAPA
GP

IL-17E is encoded by the following mRNA sequence (NCBI Accession No. AF305200 and SEQ ID NO: 9):

| | |
|---|---|
| 1 | ggcttgctga aaataaaatc aggactccta acctgctcca gtcagcctgc ttccacgagg |
| 61 | cctgtcagtc agtgcccgac ttgtgactga gtgtgcagtg cccagcatgt accaggtcag |
| 121 | tgcagagggc tgcctgaggg ctgtgctgag agggagagga gcagagatgc tgctgagggt |
| 181 | ggagggaggc caagctgcca ggtttggggc tgggggccaa gtggagtgag aaactgggat |
| 241 | cccaggggga gggtgcagat gagggagcga cccagattag gtgaggacag ttctctcatt |
| 301 | agccttttcc tacaggtg9t tgcattcttg gcaatggtca tgggaaccca cacctacagc |
| 361 | cactggccca gctgctgccc cagcaaaggg caggacacct ctgaggagct gctgaggtgg |
| 421 | agcactgtgc ctgtgcctcc cctagagcct gctaggccca accgccaccc agagtcctgt |
| 481 | agggccagtg aagatggacc cctcaacagc agggccatct ccccctggag atatgagttg |
| 541 | gacagagact tgaaccggct cccccaggac ctgtaccacg cccgttgcct gtgcccgcac |
| 601 | tgcgtcagcc tacagacagg ctcccacatg gacccccggg gcaactcgga gctgctctac |
| 661 | cacaaccaga ctgtcttcta caggcggaca tgccatggcg agaagggcac ccacaagggc |
| 721 | tactgcctgg agcgcaggct gtaccgtgtt tccttagctt gtgtgtgtgt gcggccccgt |
| 781 | gtgatgggct agccggacct gctggaggct ggtccctttt tgggaaacct ggagccaggt |
| 841 | gtacaaccac ttgccatgaa gggccaggat gcccagatgc ttggtccctg tgaagtgctg |
| 901 | tctggagcag caggatcccg ggacaggatg gggggctttg gggaaaacct gcacttctgc |
| 961 | acattttgaa aagagcagct gctgcttagg gccgccggaa gctggtgtcc tgtcattttc |
| 1021 | tctcaggaaa ggttttcaaa gttctgccca tttctggagg ccaccactcc tgtctcttcc |
| 1081 | tcttttccca tcccctgcta ccctggccca gcacaggcac tttctagata tttccccctt |
| 1141 | gctggagaag aaagagcccc tggttttatt tgtttgttta ctcatcactc agtgagcatc |
| 1201 | tactttgggt gcattctagt gtagttacta gtcttttgac atggatgatt ctgaggagga |
| 1261 | agctgttatt gaatgtatag agatttatcc aaataaatat ctttatttaa aaatgaaaaa |
| 1321 | aaaaaaaaaa aaaaa |

IL-17E is encoded by the following amino acid sequence (NCBI Accession No. AAG40848.1 and SEQ ID NO: 10):

MRERPRLGEDSSLISLFLQVVAFLAMVMGTHTYSHWPSCCPSKGQDTSEE
LLRWSTVPVPPLEPARPNRHPESCRASEDGPLNSRAISPWRYELDRDLNR
LPQDLYHARCLCPHCVSLQTGSHMDPRGNSELLYHNQTVFYRRPCHGEKG
THKGYCLERRLYRVSLACVCVRPRVMG

IL-17F, transcript 1, is encoded by the following mRNA sequence (NCBI Accession No. NM_{—}052872 and SEQ ID NO: 11):

| | |
|---|---|
| 1 | gaacacaggc atacacagga agatacatta acagaaagag cttcctgcac aaagtaagcc |
| 61 | accagcgcaa catgacagtg aagaccctgc atggcccagc catggtcaag tacttgctgc |
| 121 | tgtcgatatt ggggcttgcc tttctgagtg aggcggcagc tcggaaaatc cccaaagtag |
| 181 | gacatacttt tttccaaaag cctgagagtt gcccgcctgt gccaggaggt agtatgaagc |
| 241 | ttgacattgg catcatcaat gaaaaccagc gcgtttccat gtcacgtaac atcgagagcc |
| 301 | gctccacctc cccctggaat tacactgtca cttgggaccc caaccggtac ccctcggaag |
| 361 | ttgtacaggc ccagtgtagg aacttgggct gcatcaatgc tcaaggaaag gaagacatct |
| 421 | ccatgaattc cgttcccatc cagcaagaga ccctggtcgt ccggaggaag caccaaggct |
| 481 | gctctgtttc tttccagttg gagaaggtgc tggtgactgt tggctgcacc tgcgtcaccc |
| 541 | ctgtcatcca ccatgtgcag taagaggtgc atatccactc agctgaagaa gctgtagaaa |
| 601 | tgccactcct tacccagtgc tctgcaacaa gtcctgtctg acccccaatt ccctccactt |
| 661 | cacaggactc ttaataagac ctgcacggat ggaaacagaa aatattcaca atgtatgtgt |
| 721 | gtatgtacta cactttatat ttgatatcta aaatgttagg agaaaaatta atatattcag |
| 781 | tgctaatata ataaagtatt aataattt |

IL-17F, transcript 1, is encoded by the following amino acid sequence (NCBI Accession No. NP_{—}443104.1 and SEQ ID NO: 12)

MTVKTLHGPAMVKYLLLSILGLAFLSEAAARKIPKVGHTFFQKPESCPPV
PGGSMKLDIGIINENQRVSMSRNIESRSTSPWNYTVTWDPNRYPSEVVQA
QCRNLGCINAQGKEDISMNSVPIQQETLVVRRKHQGCSVSFQLEKVLVTV
GCTCVTPVIHHVQ

ML-1, IL-17F transcript 2, is encoded by the following mRNA sequence (NCBI Accession No. AF332389 and SEQ ID NO: 13):

| | |
|---|---|
| 1 | ggcttcagtt actagctagg ccactgagtt tagttctcag tttggcacct tgataccttt |
| 61 | aggtgtgagt gttcccattt ccaggtgagg aactgaggtg caaagagaag ccctgatccc |
| 121 | ataaaaggac aggaatgctg agttccgcca gaccatgcat ctcttgctag taggtgaggc |
| 181 | gagtctctaa ctgattgcag cgtcttctat tttccaggtc aagtacttgc tgctgtcgat |
| 241 | attggggctt gcctttctga gtgaggcggc agctcggaaa atccccaaag taggacatac |
| 301 | ttttttccaa aagcctgaga gttgcccgcc tgtgccagga ggtagtatga agcttgacat |
| 361 | tggcatcatc aatgaaaacc agcgcgtttc catgtcacgt aacatcgaga gccgctccac |
| 421 | ctccccctgg aattacactg tcacttggga ccccaaccgg tacccctcgg aagttgtaca |
| 481 | ggcccagtgt aggaacttgg gctgcatcaa tgctcaagga aaggaagaca tctccatgaa |
| 541 | ttccgttccc atccagcaag agaccctggt cgtccggagg aagcaccaag gctgctctgt |
| 601 | ttctttccag ttggagaagg tgctggtgac tgttggctgc acctgcgtca cccctgtcat |
| 661 | ccaccatgtg cagtaagagg tgcatatcca ctcagctgaa gaagctgtag aaatgccact |
| 721 | ccttacccag tgctctgcaa caagtcctgt ctgaccccca attccctcca cttcacagga |
| 781 | ctcttaataa gacctgcacg gatggaaaca taaaatattc acaatgtatg tgtgtatgta |
| 841 | ctacacttta tatttgatat ctaaaatgtt aggagaaaaa ttaatatatt cagtgctaat |
| 901 | ataataaagt attaataatg ttaaaaaaaa aaaaaaaaaa aaaaaaa |

ML-1, IL-17F transcript 2, is encoded by the following amino acid sequence (NCBI Accession No. AAL14427.1 and SEQ ID NO: 14)

MKLDIGIINENQRVSMSRNIESRSTSPWNYTVTWDPNRYPSEVVQAQCRN
LGCINAQGKEDISMNSVPIQQETLVVRRKHQGCSVSFQLEKVLVTVGCTC
VTPVIHHVQ

### DNA Compositions

DNA compositions of the invention include all polynucleotides or fragments thereof. Contemplated DNA compositions of the above methods include linearized DNA sequences. Moreover, DNA compositions include recombinant DNA sequences. In a preferred embodiment, DNA compositions include circular or linearized recombinant DNA sequences. Alternatively, or in addition, DNA compositions include the MAb composition. DNA compositions include an endogenous or exogenous sequence. In a preferred embodiment, DNA compositions include a transgene, e.g. an IL-17 transgene.

Exemplary DNA sequences contained by DNA compositions of the instant methods include, but are not limited to, a sequence encoding a polyribonucleotide, a single-stranded RNA, a double-stranded RNA, an interfering or silencing RNA, a microRNA, a polydioxyribonucleotide, a single-stranded DNA, a double-stranded DNA, a morpholino, an oligonucleotide, a polypeptide, a protein, a signaling protein, a G-protein, an enzyme, a cytokine, a chemokine, a neurotransmitter, a monoclonal antibody, a polyclonal antibody, an intrabody, a hormone, a receptor, a cytosolic protein, a membrane bound protein, a secreted protein, and/or a transcription factor.

In a preferred embodiment, the DNA composition includes at least one monoclonal antibody (MAb). MAb compositions of the invention comprise the NI-0701 expression vector or an expression vector comprising the 15C1 antibody. This expression vector is a “double gene” vector containing the heavy and light chain variable regions of antibody NI-0701 in fusion with the human IgG1 and human Lambdal constant region cassettes, respectively. The expression of each antibody chain is driven by the strong hCMV promoter. The NI-0701 vector also contains the Glutamine Synthetase (GS) gene under the control of the SV40 promoter. GS catalyses synthesis of the essential amino-acid glutamine from glutamic acid, ammonia and ATP. Selection stringency is therefore applied in absence of glutamine, and eventually in the presence of a specific GS inhibitor, methionine sulphoximine (MSX) for cell lines presenting endogenous GS activity, e.g. CHOK1SV.

### Methods

The invention provides a method of using IL-17 to enhance a property of modification of a cell with a nucleic acid, the method including the step of contacting the cell with the IL-17. In an alternative embodiment of this method, the exposure to IL-17 causes enhanced expression of the nucleic acid compared to a cell not contacted by IL-17.

The invention further provides a method of enhancing the efficacy of cell modification, including the steps of: (a) culturing one or more cells or cell line(s) in medium; (b) contacting one or more cells or cell line(s) with a nucleic acid; (c) culturing modified cells in medium to express the polypeptide encoded by the nucleic acid wherein cells are exposed to IL-17 prior to or during the contacting step; and wherein one or more cell lines expressing one or more polypeptides is generated that demonstrates an enhanced property of transfection.

The above methods encompass a cell or cell lines under selective pressure. In one embodiment, the selective pressure is applied by growing transfected cells in a medium comprising a specific glutamine synthetase inhibitor, wherein transfected cells survive, and untransfected cells die. In a preferred embodiment, the specific glutamine synthetase inhibitor is methionine sulphoximine (MSX). Increase of selection pressure on the cell selection, for example, by increasing the concentration of MSX in the medium (e.g., above 50 μM) in the presence of an IL-17 cytokine, preferably IL-17F, increased the productivity. Increasing selective pressure in the absence of an IL-17 cytokine, preferably IL-17F, resulted in the absence of clones. Thus, the addition of IL-17F and increasing the selective pressure increases the productivity of the methods provided herein.

When selective pressure is applied, the modification is semi-stable. Alternatively, when selective pressure is applied, the modification is stable. In another embodiment the modified cells are grown in the absence of selective pressure, and therefore, the modification is transient.

Cells or cell line(s) of the above methods express at least one IL-17 receptor. Exemplary IL-17 receptors (IL-17Rs) include, but are not limited to, IL-17RA, IL-17RB, IL-17RC, IL-17RD, and IL-17RE. In one embodiment, cells or cell lines(s) include Th17 cells which secrete an IL-17 polypeptide. Exemplary IL-17 polypeptides, or cytokines encompassed by the invention include, but are not limited to, IL-17A, IL-17B, IL-17C, IL-17D, IL-17E, or IL-17F. In a preferred embodiment, an IL-17F cytokine is used.

Contemplated cells or cell line(s) of the invention include eukaryotic cells, including for example, mammalian cells. In some embodiments, the cells or cell line(s) include human cells. In an alternate embodiment, the invention includes stem cells, totipotent cells, multipotent cells, or pluripotent cells. In another embodiment, the invention includes immortalized cells. In a further embodiment, primary cells are used in culture. In another alternative embodiment, hybridoma cells are used in culture. The invention includes the use of all of the above cell types or cell populations in isolation or as mixtures. The above cell types are used simultaneously or sequentially. Any combination of the above cell types or cell populations is contemplated and encompassed by the present invention.

The above methods include multiple cell modification techniques. Exemplary cell modification methods include, but are not limited to, electroporation, heat shock, magnetofection, microinjection, gene gun, endocytosis, vesicle fusion, and lipofection. Alternatively, or in addition, cells are modified using any of a variety of viral-based gene delivery systems including, for example, parvovirus, adenovirus, retrovirus, lentivirus, and herpesvirus-based vectors. Alternatively, or in addition, nucleic acids of the invention are bound, coupled, operably linked, fused, or tethered, to compounds that facilitate transportation of these nucleic acids into a cell or cell lines. In one embodiment, a nucleic acid is bound to a cationic polymer. In another embodiment, a nucleic acid is coupled to a nanoparticle. In a third embodiment, a nucleic acid is bound to calcium phosphate.

The above methods enhance one or more properties of cell modification. Exemplary properties which are enhanced include, but are not limited to, increased efficiency, increased selection rate, increased cell growth, increased appearance speed of selected cells, increased number of selected cell lines, increased doubling time of selected cells, increased cell viability, reduced sensitivity to medium depletion, or increased cell line stability.

The above methods enhance expression of the nucleic acid by cell contact with IL-17. Exemplary properties of nucleic acid expression include, but are not limited to, increased specific production rate of monoclonal antibody (MAb), increased MAb titer, increased product quality, correlation of IL-17 expression with MAb titer, increased expression following transient modification of transfection-resistant cell-lines, or increased transgene productivity, increased incorporation of exogenous DNA into genomic sequence, increased retention of exogenous DNA, increased uptake of DNA, or increased expression of exogenous DNA.

The invention provides an IL-17 composition including at least one expression vector containing one or more IL-17 cytokine polynucleotide sequence(s) under the control of a first promoter sequence and a reporter gene downstream of the IL-17 cytokine sequence under the control of the first promoter sequence, wherein the IL-17 cytokine and reporter gene sequences are separated by an internal ribosome entry site (IRES) sequence, and wherein the expression vector further comprises a selection gene under the control of a second promoter sequence.

The IL-17 cytokine sequence is a mammalian sequence. Exemplary mammalian sources of IL-17 sequence include, but are not limited to, mouse, hamster, guinea pig, rat, pig, cat, dog, horse, and non-human primates (e.g. chimp). In a preferred embodiment, the IL-17 cytokine sequence is either a rat sequence or a human sequence. All members of the IL-17 cytokine family are contemplated including IL-17A, IL-17B, IL-17C, IL-17D, IL-17E, or IL-17F. In a preferred embodiment, the IL-17 cytokine sequence is one or more isoform(s) of IL-17F.

In some embodiments, IL-17 compositions also include a reporter gene. Contemplated reporter genes encode for polypeptides that provide a detectable signal. Alternatively, or in addition, reporter signals are bound to DNA compositions. Exemplary detectable signals are produced by luciferase (an enzyme that catalyzes a reaction with luciferin), fluorescent proteins (green, blue, red, yellow, or cyan), β-galatosidase, magnetic or paramagnetic molecules, or lipophilic dye (e.g. DiI, DiD, or DiO). The reporter gene is, for example, green fluorescent protein (GFP). These IL-17 compositions that include a reporter gene are useful, e.g., as diagnostic and/or research tools.

The invention further provides a monoclonal antibody (MAb) composition including at least one expression vector containing a polynucleotide sequence encoding an antibody heavy chain (variable and constant domains) and a polynucleotide sequence encoding an antibody light chain (variable and constant domains) both under the control of their own promoter sequence, wherein the expression vector further contains a selection gene under the control of a third promoter sequence. In a preferred embodiment, the heavy chain and light chain sequences encode the 15C1 antibody (described in U.S. Ser. No. 11/151,916, published as US 2008-0050366 A1, and U.S. Ser. No. 11/301,373, published as US 2006-0165686 A1, the contents of each of which are incorporated herein in their entirety) Furthermore, the invention provides humanized, chimeric, and recombinant monoclonal antibodies and fragments thereof, as well as scaffold molecules and other molecules that include an IgG or IgG-like domain. Contemplated monoclonal antibodies include a single or double chain and fragments thereof. Alternatively, or in additional, monoclonal antibodies of the invention are intrabodies and fragments thereof.

The IL-17 and MAb compositions of the invention include promoter elements to regulate expression of DNA sequences. These promoter elements are wild type. Alternatively, or in addition, promoter elements are engineered or chosen to perform certain functions. For instance, a promoter is engineered or chosen to induce strong expression of DNA compositions. In another example, a promoter is engineered or chosen to be inducible by addition of a chemical or compound to the culture media. For example, an inducible reporter is activated and repressed by the addition and removal, respectively, of tetracycline to and from the culture media. In another example, a promoter is constitutively active. In one preferred embodiment, the first promoter sequence is hCMV. In another embodiment the first promoter is a cellular promoter. In one preferred embodiment, the first promoter is elongation factor 1 alpha (EF-1α). In another preferred embodiment, the second promoter sequence is simian virus 40 (SV40). Other art-recognized mammalian expression vectors and viral promoter sequences are contemplated and encompassed by the invention; see Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

The IL-17 and MAb compositions of the invention include at least one selection gene. Selection genes of the invention encode for an element that is required for survival under certain culture conditions. Exemplary selection genes include, but are not limited to, those genes whose products provide antibiotic resistance, essential nutrients, essential enzymes, metabolic enzymes, and anti-apoptotic/autophagic elements. In a preferred embodiment, the selection gene encodes for glutamine synthase

The invention also provides a method of using an IL-17 composition to enhance a property of transfection and enhance expression of one or more exogenous gene(s) within one or more cell lines including inserting a DNA composition into one or more cell lines wherein the IL-17 composition contacts one or more cells.

In one embodiment, the IL-17 composition of the above methods contacts one or more cells prior to insertion of the DNA composition. Alternatively, or in addition to the first embodiment, the IL-17 composition contacts one or more cells during insertion of the DNA composition. In a further embodiment, and further in addition to the previous embodiments, the IL-17 composition contacts one or more cells following insertion of the DNA composition. In another embodiment, the IL-17 composition contacts one or more cells continuously.

The IL-17 composition of the above methods contacts one or more cells on the extracellular surface of the cell. Alternatively, or in addition, the IL-17 composition contacts one or more cells on the intracellular surface of the cell. In another embodiment, the DNA composition of the invention comprises one or more sequences encoding an IL-17 cytokine.

In a preferred embodiment, cell lines of the above methods are under selective pressure and the transfection is semi-stable or stable. Alternatively, cell lines are not placed under selective pressure and the transfection is transient. Transfection methods encompassed by the present invention include, but are not limited to, electroporation, heat shock, magnetofection, microinjection, gene gun, viral transduction, endocytosis, vesicle fusion, calcium phosphate, liposomes, and mediation by cationic polymer.

The IL-17 composition is transfected into one or more cell lines. Moreover, the IL-17 composition is transfected simultaneously or sequentially with the DNA composition. Furthermore, the IL-17 composition is an exogenous sequence co-expressed with one or more exogenous gene(s).

Alternatively, or in addition, the IL-17 composition is present in the transfection medium before, during, or following transfection. The IL-17 composition binds one or more extracellular proteins associated with a cell expressing one or more exogenous gene(s). The IL-17 composition binds one or more membrane-spanning proteins associated with a cell expressing one or more exogenous gene(s). In one embodiment, the IL-17 composition is endocytosed by one or more cell line(s) expressing one or more exogenous gene(s). Thus, the IL-17 composition binds one or more intracellular proteins associated with a cell expressing one or more exogenous gene(s).

The invention further provides a method of enhancing the efficacy of semi-stable transfection, including the steps of: (a) culturing one or more cell line(s) in medium; (b) mixing the cell line(s) with one or more DNA compositions; (c) transporting one or more DNA compositions across the plasma membranes of at least one cell line; (d) culturing transfected cells in medium under selective pressure; and (e) allowing transfected cells to express polypeptides encoded by the transfected DNA compositions under selective pressure; wherein a mixture of cell lines expressing one or more polypeptides is generated that demonstrates an enhanced property of transfection.

The invention also provides a method of enhancing the efficacy of stable transfection, including the steps of: (a) culturing one or more cell line(s) in medium; (b) mixing the cell line(s) with one or more DNA compositions; (c) transporting one or more DNA compositions across the plasma membranes of at least one cell line; (d) culturing transfected cells in medium under selective pressure; and (e) allowing transfected cells to express polypeptides encoded by the transfected DNA compositions under selective pressure; wherein an isolated cell line expressing one or more polypeptides is generated that demonstrates an enhanced property of transfection.

DNA compositions of the above semi-stable and stable transfection methods include at least one sequence that encodes for an IL-17. In an alternative embodiment, the culture medium includes at least one IL-17 polypeptide. In another embodiment, the cell line(s) express at least one IL-17 receptor. Exemplary IL-17 receptors (IL-17Rs) encompassed by the invention and present methods include, but are not limited to, IL-17RA, IL-17RB, IL-17RC, IL-17RD, and IL-17RE. In a further embodiment, the cell lines comprise Th17, neutrophils, macrophages and γ-T cells, which secrete an IL-17 polypeptide

IL-17 compositions of the above methods include an IL-17 polypeptide that is wild type or mutant. Functionally, IL-17 compositions of the above methods include an IL-17 polypeptide that is active or inactive. Alternatively, IL-17 compositions of the above methods include an inactive IL-17 mutant. Exemplary IL-17 polypeptides include all members of the IL-17 family. The IL-17 cytokine family includes, but is not limited to, IL-17A, IL-17B, IL-17C, IL-17D, IL-17E, or IL-17F. In a preferred embodiment, IL-17 compositions of the above methods contain an IL-17F polypeptide. IL-17F exists as one of two isoforms, both of which are contemplated and encompassed by the compositions and methods of the invention. IL-17F isoform 2 is also known as ML-1, and is encompassed by the invention.

Cell line(s) of the above methods include eukaryotic cells including, for example, mammalian cells. In some embodiments, the cells or cell line(s) include human cells. Cell line(s) include humanized cells and hybridomas and immortalized primary cells such as, for example, lymphocyte B. In one embodiment, cell lines include stem cells, totipotent cells, multipotent cells, or pluripotent cells. Cell line(s) include embryonic, fetal, neonatal, perinatal, childhood, or adult cells. In another embodiment, cell lines include immortalized cells. Cell lines have endothelial, mesenchymal, or mesodermal origin. In an alternate embodiment, cell lines include primary cells in culture. Furthermore, cell lines include hybridoma cells in culture.

Cell line(s) include smooth or striated muscle cells. In one embodiment, cell line(s) include cardiac cells.

Encompassed cell lines include an immune cell that is a hematopoietic cell, a lymphoid cell, a myeloid cell, a lymphocyte precursor, a B cell precursor, a T cell precursor, a lymphocyte, a B cell, a T cell, a plasma cell, a monocyte, a macrophage, a neutrophil, an eosinophil, a basophil, a natural killer cell, a mast cell, or a dendritic cell.

Encompassed cell lines include a neural cell that is a neuron, a basket cell, a betz cell, a medium spiny neuron, a purkinje cell, a pyramidal cell, a projection neuron, a renshaw cell, a granule cell, a motoneuron, an excitatory neuron, an inhibitory neuron, a spindle neuron, a neural precursor, a neural stem cell, an interneuron, a glial cell, a radial glial cell, an astrocyte/astroglia (type 1 or type 2), an oligodendrocyte, a Schwann cell, or a Bergmann glial cell. Contemplated cell lines also include epithelial and endothelial cells of all types.

Cell line(s) of the present invention also include all types of cancer cells. Cancer cells encompassed by the invention are derived from the following exemplary conditions which, include, but are not limited to, acute lymphoblastic leukemia, acute myeloid leukemia, adrenocortical carcinoma, adrenocortical carcinoma, AIDS-related cancers, AIDS-related lymphoma, anal cancer, appendix cancer, childhood cerebellar astrocytoma, childhood cerebral astrocytoma, basal cell carcinoma, skin cancer (non-melanoma), extrahepatic bile duct cancer, bladder cancer, bone cancer, osteosarcoma and malignant fibrous histiocytoma, brain tumor, brain stem glioma, cerebellar astrocytoma, cerebral astrocytoma/malignant glioma, ependymoma, medulloblastoma, supratentorial primitive neuroectodermal tumors, visual pathway and hypothalamic glioma, breast cancer, bronchial adenomas/carcinoids, carcinoid tumor, gastrointestinal, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, mycosis fungoides, Séary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach)cancer, gastrointestinal carcinoid tumor, gastrointestinal stromal tumor (GIST), germ cell tumor, ovarian germ cell tumor, gestational trophoblastic tumor glioma, head and neck cancer, hepatocellular (liver) cancer, Hodgkin lymphoma, hypopharyngeal cancer, intraocular melanoma, islet cell tumors (endocrine pancreas), Kaposi Sarcoma, kidney (renal cell) Cancer, kidney cancer, laryngeal cancer, acute lymphoblastic leukemia, acute myeloid leukemia, chronic lymphocytic leukemia, chronic myelogenous leukemia, hairy cell leukemia, lip and oral cavity cancer, liver cancer, non-small cell lung cancer, small cell lung cancer, AIDS-related lymphoma, non-Hodgkin lymphoma, primary central nervous system lymphoma, Waldenström macroglobulinemia, medulloblastoma, melanoma, intraocular (eye) melanoma, merkel cell carcinoma, mesothelioma malignant, mesothelioma, metastatic squamous neck cancer, mouth cancer, multiple endocrine neoplasia syndrome, mycosis fungoides, myelodysplastic syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, paranasal sinus and nasal cavity cancer, parathyroid cancer, penile cancer, pharyngeal cancer, pheochromocytoma, pineoblastoma and supratentorial primitive neuroectodermal tumors, pituitary Tumor, plasma cell neoplasm/multiple myeloma, pleuropulmonary blastoma, prostate Cancer, rectal Cancer, renal pelvis and ureter, transitional cell cancer, retinoblastoma, rhabdomyosarcoma, salivary gland cancer, ewing family of sarcoma tumors, Kaposi Sarcoma, soft tissue sarcoma, skin cancer (nonmelanoma), skin cancer (melanoma), merkel cell skin carcinoma, small intestine cancer, soft tissue sarcoma, squamous cell carcinoma, stomach (gastric) cancer, supratentorial primitive neuroectodermal tumors, testicular Cancer, throat Cancer, thymoma, thymoma and thymic carcinoma, thyroid cancer, transitional cell cancer of the renal pelvis and ureter, gestational trophoblastic tumor, urethral cancer, endometrial uterine cancer, uterine sarcoma, vaginal cancer, vulvar cancer, and Wilms Tumor.

Preferred cells used in the above methods are any rodent cell line, including for example, CHOK1SV cells or CHO—S cells. In embodiments where CHOK1SV or CHO—S cells are used, the preferred culture medium in the above methods is CD-CHO supplemented with 6 mM L-glutamine. Other cells and cell lines include cells and cell lines used in the Boehringer Ingelheim's High Expression System (BI-HEX®), including, for example, CHO-DG44 cells.

DNA compositions of the above methods are either transported across cell membranes or inserted by electroporation, heat shock, magnetofection, or gene gun. Alternatively, DNA compositions of the above methods are either transported across cell membranes or inserted by viral transduction. Furthermore, DNA compositions of the above methods are either transported across cell membranes or inserted by endocytosis, vesicle fusion, or liposomes. DNA compositions of the above methods include one or more DNA sequences bound to a cationic polymer to increase probability of uptake by one or more cell lines. Exemplary cationic polymers include, but are not limited to, polylysine, polyamidamine, and polyethylenimine. Alternatively, DNA compositions of the above methods include one or more DNA sequences coupled to a nanoparticle. Contemplated nanoparticles include inert solid materials including, but not limited to, gold, to enable transfection by gene gun. Furthermore, DNA compositions of the above methods include one or more DNA sequences bound to calcium phosphate to enable uptake by one or more cell lines. DNA compositions further include one or more DNA sequences encapsulated by a virus to enable viral transformation. DNA compositions further include one or more DNA sequences incorporated into or associated with liposomes for lipofection. For example, lipofection is accomplished using Lipofectamine (Invitrogen).

DNA compositions of the above methods include linearized DNA sequences. Moreover, DNA compositions include recombinant DNA sequences. In a preferred embodiment, DNA compositions include linearized recombinant DNA sequences. Alternatively, or in addition, DNA compositions include a MAb composition. DNA compositions include an endogenous or exogenous sequence. In a preferred embodiment, DNA compositions include a transgene, e.g. an IL-17 transgene.

Exemplary DNA sequences contained by DNA compositions of the instant methods include, but are not limited to, a sequence encoding a polyribonucleotide, a single-stranded RNA, a double-stranded RNA, an interfering or silencing RNA, a microRNA, a polydioxyribonucleotide, a single-stranded DNA, a double-stranded DNA, a morpholino, an oligonucleotide, a polypeptide, a protein, a signaling protein, a G-protein, an enzyme, a cytokine, a chemokine, a neurotransmitter, a monoclonal antibody, a polyclonal antibody, an intrabody, a hormone, a receptor, a cytosolic protein, a membrane bound protein, a secreted protein, or a transcription factor.

### DEFINITIONS

Unless otherwise defined, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo- or polynucleotide chemistry and hybridization described herein are those well known and commonly used in the art. Standard techniques are used for recombinant DNA and oligonucleotide synthesis, as well as tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification. See e.g., Sambrook et al. Molecular Cloning: A Laboratory Manual (2d ed., Cold Spring Harbor Laboratory Press, Cold Spring Harbor, N.Y. (1989)). The nomenclatures utilized in connection with, and the laboratory procedures and techniques of analytical chemistry, synthetic organic chemistry, and medicinal and pharmaceutical chemistry described herein are those well known and commonly used in the art. Standard techniques are used for chemical syntheses, chemical analyses, pharmaceutical preparation, formulation, delivery and treatment of patients.

As utilized in accordance with the present disclosure, the following terms, unless otherwise indicated, shall be understood to have the following meanings:

The term “polynucleotide” as referred to herein means a polymeric boron of nucleotides of at least 10 bases in length, either ribonucleotides or deoxynucleotides or a modified form of either type of nucleotide. The term includes single and double stranded forms of DNA.

The term “polypeptide” is used herein as a generic term to refer to native protein, fragments, or mutants of a polypeptide sequence. Hence, native protein fragments, and mutants are species of the polypeptide genus. Preferred polypeptides in accordance with the invention comprise cytokines and antibodies.

As used herein, the term “antibody” refers to immunoglobulin molecules and immunologically active portions of immunoglobulin (Ig) molecules, i.e., molecules that contain an antigen binding site that specifically binds (immunoreacts with) an antigen. Such antibodies include, but are not limited to, polyclonal, monoclonal, chimeric, single chain, F_{ab}, F_{ab}' and F_{(ab′)2} fragments, and antibodies in an F_{ab} expression library. By “specifically bind” or “immunoreacts with” is meant that the antibody reacts with one or more antigenic determinants of the desired antigen and does not react (i.e., bind) with other polypeptides or binds at much lower affinity (K_{d}>10⁻⁶) with other polypeptides.

The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. Human light chains are classified as kappa and lambda light chains. Heavy chains are classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989)). The variable regions of each light/heavy chain pair form the antibody binding site.

The term “monoclonal antibody” (MAb) or “monoclonal antibody composition”, as used herein, refers to a population of antibody molecules that contain only one molecular species of antibody molecule consisting of a unique light chain gene product and a unique heavy chain gene product. In particular, the complementarity determining regions (CDRs) of the monoclonal antibody are identical in all the molecules of the population. MAbs contain an antigen binding site capable of immunoreacting with a particular epitope of the antigen characterized by a unique binding affinity for it.

In general, antibody molecules obtained from humans relate to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain.

The term “intrabody” as used herein shall mean a polypeptide comprising an intracellular antibody. Intrabodies are not secreted. Intrabodies bind intracellular targets including polynucleotide and polypeptide sequences. Intrabodies enter all cellular compartments.

The term “fragments thereof” as used herein shall mean a segment of a polynucleotide sequence or polypeptide sequence that is less than the length of the entire sequence. Fragments as used herein comprised functional and non-functional regions. Fragments from different polynucleotide or polypeptide sequences are exchanged or combined to create a hybrid or “chimeric” molecule. Fragments are also used to modulate polypeptide binding characteristics to either polynucleotide sequences or to other polypeptides.

The term “promoter sequence” as used herein shall mean a polynucleotide sequence comprising a region of a gene at which initiation and rate of transcription are controlled. A promoter sequence comprises an RNA polymerase binding site as well as binding sites for other positive and negative regulatory elements. Positive regulatory elements promote the expression of the gene under control of the promoter sequence. Negative regulatory elements repress the express of the gene under control of the promoter sequence. Promoter sequences used herein are found either upstream or internal to the gene being regulated. Specifically, the term “first promoter sequence” versus “second promoter sequence” refers to the relative position of the promoter sequence within the expression vector. The first promoter sequence is upstream of the second promoter sequence.

The term “selection gene” as used herein shall mean a polynucleotide sequence encoding for a polypeptide that is necessary for the survival of the cell in the given culture conditions. If a cell has successfully incorporated the expression vector carrying the gene of interest, along with the selection gene, that cell will produce an element that will allow it to selectively survive under hostile culture conditions. “Selected” cells are those which survive under selective pressure and must have incorporated the expression vector. The term “selective pressure” as used herein shall mean the addition of an element to cell culture medium that inhibits the survival of cells not receiving the DNA composition.

The term “endogenous gene” as used herein shall mean a gene encompassed within the genomic sequence of a cell. The term “exogenous gene” as used herein shall mean a gene not encompassed within the genomic sequence of a cell. Exogenous genes are introduced into cells by the instant methods. The term “transgene” as used herein shall mean a gene that has been transferred from one organism to another.

The term “transfection” as used herein shall mean the transportation across the cell membrane or insertion of one or more DNA compositions into a cell. “Stable transfection” as used herein shall mean the generation, under selective pressure, of isolated protein-expressing cell lines. “Semi-stable transfection” as used herein shall mean the generation, under selective pressure, of a mixture of protein-expressing cell lines. “Transient transfection” as used herein shall mean the generation, without selective pressure, of protein-expressing cell lines. Stable and semi-stable transfections may lead to incorporation of transfected sequences into the genome due to selective pressure. Transient transfections do not lead to genomic incorporation of transfected sequences and typically retain these sequences for a shorter period of time. The term “transfection-resistant” as used herein shall mean transfected with low efficiency or success using known methods.

The term “enhanced property” as used herein shall mean a property superior with respect to that same parameter when measured in the absence of IL-17.

The term “reporter gene” as used herein shall mean a polynucleotide sequence encoding for a polypeptide that creates a physical change in those cells which incorporate the expression vector, and, thus, the gene of interest. Physical changes are often color changes or fluorescence.

The term “internal ribosome entry site (IRES)” as used herein shall mean a polynucleotide sequence that allows for translation initiation in the middle of a messenger RNA (mRNA) sequence, a process that does not naturally occur in eukaryotic cells. Placement of an IRES segment between two open reading frames in a eukaryotic mRNA molecule (referred to as a bicistronic mRNA), drives translation of the downstream protein coding region independently of the 5′-cap structure bound to the 5′ end of the mRNA molecule. The result is that both proteins are produced in the cell.

As used herein, the twenty conventional amino acids and their abbreviations follow conventional usage. See Immunology—A Synthesis (2nd Edition, E. S. Golub and D. R. Gren, Eds., Sinauer Associates, Sunderland Mass. (1991)). Stereoisomers (e.g., D-amino acids) of the twenty conventional amino acids, unnatural amino acids such as α-, α-disubstituted amino acids, N-alkyl amino acids, lactic acid, and other unconventional amino acids may also be suitable components for polypeptides of the present invention. Examples of unconventional amino acids include: 4 hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, σ-N-methylarginine, and other similar amino acids and imino acids (e.g., 4-hydroxyproline). In the polypeptide notation used herein, the lefthand direction is the amino terminal direction and the righthand direction is the carboxy-terminal direction, in accordance with standard usage and convention.

Similarly, unless specified otherwise, the lefthand end of single-stranded polynucleotide sequences is the 5′ end the lefthand direction of double-stranded polynucleotide sequences is referred to as the 5′ direction. The direction of 5′ to 3′ addition of nascent RNA transcripts is referred to as the transcription direction sequence regions on the DNA strand having the same sequence as the RNA and which are 5′ to the 5′ end of the RNA transcript are referred to as “upstream sequences”, sequence regions on the DNA strand having the same sequence as the RNA and which are 3′ to the 3′ end of the RNA transcript are referred to as “downstream sequences”.

Silent or conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine valine, glutamic-aspartic, and asparagine-glutamine.

Silent or conservative replacements are those that take place within a family of amino acids that are related in their side chains. Genetically encoded amino acids are generally divided into families: (1) acidic amino acids are aspartate, glutamate; (2) basic amino acids are lysine, arginine, histidine; (3) non-polar amino acids are alanine, valine, leucine, isoleucine, proline, phenylalanine, methionine, tryptophan, and (4) uncharged polar amino acids are glycine, asparagine, glutamine, cysteine, serine, threonine, tyrosine. The hydrophilic amino acids include arginine, asparagine, aspartate, glutamine, glutamate, histidine, lysine, serine, and threonine. The hydrophobic amino acids include alanine, cysteine, isoleucine, leucine, methionine, phenylalanine, proline, tryptophan, tyrosine and valine. Other families of amino acids include (i) serine and threonine, which are the aliphatic-hydroxy family; (ii) asparagine and glutamine, which are the amide containing family; (iii) alanine, valine, leucine and isoleucine, which are the aliphatic family; and (iv) phenylalanine, tryptophan, and tyrosine, which are the aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Assays are described in detail herein. Fragments or analogs of antibodies or immunoglobulin molecules can be readily prepared by those of ordinary skill in the art. Preferred amino- and carboxy-termini of fragments or analogs occur near boundaries of functional domains. Structural and functional domains can be identified by comparison of the nucleotide and/or amino acid sequence data to public or proprietary sequence databases. Preferably, computerized comparison methods are used to identify sequence motifs or predicted protein conformation domains that occur in other proteins of known structure and/or function. Methods to identify protein sequences that fold into a known three-dimensional structure are known. Bowie et al. Science 253:164 (1991). Thus, the foregoing examples demonstrate that those of skill in the art can recognize sequence motifs and structural conformations that may be used to define structural and functional domains in accordance with the invention.

A silent or conservative amino acid substitution should not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et at. Nature 354:105 (1991).

Other chemistry terms herein are used according to conventional usage in the art, as exemplified by The McGraw-Hill Dictionary of Chemical Terms (Parker, S., Ed., McGraw-Hill, San Francisco (1985)).

### EXAMPLES

### Example 1

### The NI-0701 Double Gene Expression Vector

The NI-0701 expression vector is a “double gene” vector containing the heavy and light chain variable regions of antibody NI-0701 in fusion with the human IgG1 and human Lambdal constant region cassettes, respectively. The expression of each antibody chain is driven by the strong hCMV promoter. The NI-0701 vector also contains the Glutamine Synthetase (GS) gene under the control of the SV40 promoter. GS catalyses synthesis of the essential amino-acid glutamine from glutamic acid, ammonia and ATP. Selection stringency is therefore applied in absence of glutamine, and eventually in the presence of a specific GS inhibitor, methionine sulphoximine (MSX) for cell lines presenting endogenous GS activity, e.g. CHOK1SV.

The NI-0701 Heavy Chain, Variable Domain, is encoded by the following nucleic acid sequence (SEQ ID NO: 15):

CAGGTGCAGCTGGTGCAGTCTGGGGCTGAGGTGAAGAAGCCTGGGGCCTC
AGTGAAGGTTTCCTGCAAGGTTTCCGGATACACCCTCACTGAGTTCGCCA
TGCACTGGGTGCGACAGGCTCCTGGAAAAGGGCTTGAGTGGATGGGAGGT
TTTGTTCCTGAAGATGGTGAGACAATCTACGCGCAGAAGTTCCAGGGCAG
AGTCACCATGACCGAGGACACATCTACAGACACAGCCTACATGGAGCTGA
GCAGCCTGAGATCTGAGGACACGGCCGTGTATTACTGTGCAACAGATCCC
CTGTATGAGGGTTCGTTTTCTGTTTGGGGGCAGGGGACCACGGTCACCGT
CTCGAGT

The NI-0701 Heavy Chain, Variable Domain, is encoded by the following amino acid sequence (SEQ ID NO: 16)

QVQLVQSGAEVKKPGASVKVSCKVSGYTLTEFAMHWVRQAPGKGLEWMGG
FVPEDGETIYAQKFQGRVTMTEDTSTDTAYMELSSLRSEDTAVYYCATDP
LYEGSFSVWGQGTTVTVSS

The NI-0701 Light Chain, Variable Domain is encoded by the following nucleic acid sequence (SEQ ID NO: 17):

TCCTATGTGCTGACTCAGCCACCCTCGGTGTCAGTGGCCCCAGGACAGAC
GGCCAGGATTACCTGTGGGGGAAACAACATTGAAAGTAAAAGTGTGCACT
GGTACCAGCAGAAGCCAGGCCAGGCCCCTGTGCTGGTGGTCTATGATGAT
AGCGACCGGCCCTCAGGGATCCCTGAGCGATTCTCTGGCTCCAACTCTGG
GAACACGGCCACCCTGACCATCAGCAGGGTCGAAGCCGGGGATGAGGCCG
ACTATTACTGTCAGGTGTGGGATAGTAATACTGATCATTGGGTGTTCGGC
GGAGGGACCAAGCTCACCGTCCTA

The NI-0701 Light Chain, Variable Domain, is encoded by the following amino acid sequence (SEQ ID NO: 18)

SYVLTQPPSVSVAPGQTARITCGGNNIESKSVHWYQQKPGQAPVLVVYDD
SDRPSGIPERFSGSNSGNTATLTISRVEAGDEADYYCQVWDSNTDHWVFG
GGTKLTVL

The NI-0701 Heavy Chain is encoded by the following amino acid sequence (SEQ ID NO: 19)

QVQLVQSGAEVKKPGASVKVSCKVSGYTLTEFAMHWVRQAPGKGLEWMGG
FVPEDGETIYAQKFQGRVTMTEDTSTDTAYMELSSLRSEDTAVYYCATDP
LYEGSFSVWGQGTTVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKRVEPKSCDKTHTCPPCPAPELLGGPSVFLFPPKPK
DTLMISRTPEVTCVVVDVSHEDPEVKFNWYVDGVEVHNAKTKLPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK

The NI-0701 Light Chain is encoded by the following amino acid sequence (SEQ ID NO: 20)

SYVLTQPPSVSVAPGQTARITCGGNNIESKSVHWYQQKPGQAPVLVVYDD
SDRPSGIPERFSGSNSGNTATLTISRVEAGDEADYYCQVWDSNTDHWVFG
GGTKLTVLGQPKAAPSVTLFPPSSEELQANKATLVCLISDFYPGAVTVAW
KADSSPVKAGVETTTPSKQSNNKYAASSYLSLTPEQWKSHRSYSCQVTHE
GSTVEKTVAPTECS

### Example 2

### Generation of the IL-17 Expression Vector

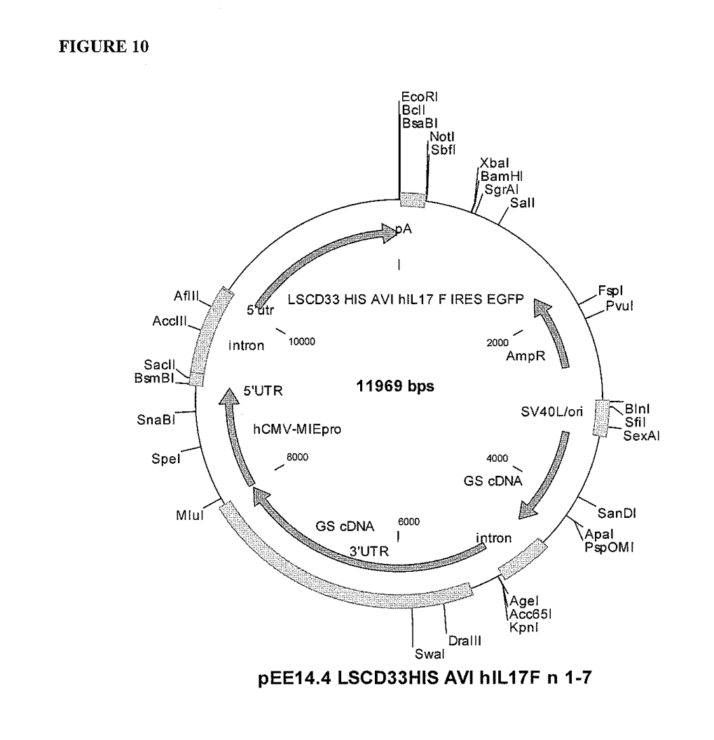

The human interleukin 17F (IL-17F or hIL-17F) and 17A (IL-17A or hIL-17A) and rat interleukin IL-17F (rat IL-17F or rIL-17F), were isolated from human or rat cDNA and subsequently sub-cloned in an expression vector under the control of the hCMV promoter. GFP was cloned downstream of the hIL-17 cDNA as a second cistron under the control of the same CMV promoter. The two cistrons (IL-17 and GFP) were separated by viral internal ribosome entry site (IRES) to allow for translation of the second (GFP) cistron. The vector also contained the GS gene under the control of the SV40 promoter for selection of transfected cells in glutamine-free medium using MSX. FIG. 10 is a map of the IL-17 expression vector.

The IL-17 Expression Vector, is encoded by the following nucleic acid sequence (SEQ ID NO: 21):

GAATTCATTGATCATAATCAGCCATACCACATTTGTAGAGGTTTTACTTG
CTTTAAAAAACCTCCCACACCTCCCCCTGAACCTGAAACATAAAATGAAT
GCAATTGTTGTTGTTAACTTGTTTATTGCAGCTTATAATGGTTACAAATA
AAGCAATAGCATCACAAATTTCACAAATAAAGCATTTTTTTCACTGCATT
CTAGTTGTGGTTTGTCCAAACTCATCAATGTATCTTATCATGTCTGGCGG
CCGCGACCTGCAGGCGCAGAACTGGTAGGTATGGAAGATCCCTCGAGATC
CATTGTGCTGGCGGTAGGCGAGCAGCGCCTGCCTGAAGCTGCGGGCATTC
CCAGTCAGAAATGAGCGCCAGTCGTCGTCGGCTCTCGGCACCGAAGTGCT
ATGATTCTCCGCCAGCATGGCTTCGGCCAGTGCGTCGAGCAGCGCCCGCT
TGTTCCTGAAGTGCCAGTAAAGCGCCGGCTGCTGAACCCCCAACCGTTCC
GCCAGTTTGCGTGTCGTCAGACCGTCTACGCCGACCTCGTTCAACAGGTC
CAGGGCGGCACGGATCACTGTATTCGGCTOCAACTTTGTCATGCTTGACA
CTTTATCACTGATAAACATAATATGTCCACCAACTTATCAGTGATAAAGA
ATCCGCGCCAGCACAATGGATCTCGAGGTCGAGGGATCTCTAGAGGATCC
TCTACGCCGGACGCATCGTGGCCGGCATCACCGGCGCCACAGGTGCGGTT
GCTGGCGCCTATATCGCCGACATCACCGATGGGGAAGATCGGGCTCGCCA
CTTCGGGCTCATGAGCGCTTGTTTCGGCGTGGGTATGGTGGCAGGCCCCG
TGGCCGGGGGACTGTTGGGCGCCATCTCCTTGCATGCACCATTCCTTGCG
GCGGCGGTGCTCAACGGCCTCAACCTACTACTGGGCTGCTTCCTAATGCA
GGAGTCGCATAAGGGAGAGCGTCGACCTCGGGCCGCGTTGCTGGCGTTTT
TCCATAGGCTCCGCCCCCCTGACGAGCATCACAAAAATCGACGCTCAAGT
CAGAGGTGGCGAAACCCGACAGGACTATAAAGATACCAGGCGTTTCCCCC
TGGAAGCTCCCTCGTGCGCTCTCCTGTTCCGACCCTGCCGCTTACCGGAT
ACCTGTCCGCCTTTCTCCCTTCGGGAAGCGTGGCGCTTTCTCATAGCTCA
CGCTGTAGGTATCTCAGTTCGGTGTAGGTCGTTCGCTCCAAGCTGGGCTG
TGTGCACGAACCCCCCGTTCAGCCCGACCGCTGCGCCTTATCCGGTAACT
ATCGTCTTGAGTCCAACCCGGTAAGACACGACTTATCGCCACTGGCAGCA
GCCACTGGTAACAGGATTAGCAGAGCGAGGTATGTAGGCGGTGCTACAGA
GTTCTTGAAGTGGTGGCCTAACTACGGCTACACTAGAAGAACAGTATTTG
GTATCTGCGCTCTGCTGAAGCCAGTTACCTTCGGAAAAAGAGTTGGTAGC
TCTTGATCCGGCAAACAAACCACCGCTGGTAGCGGTGGTTTTTTTGTTTG
CAAGCAGCAGATTACGCGCAGAAAAAAAGGATCTCAAGAAGATCCTTTGA
TCTTTTCTACGGGGTCTGACGCTCAGTGGAACGAAAACTCACGTTAAGGG
ATTTTGGTCATGAGATTATCAAAAAGGATCTTCACCTAGATCCTTTTAAA
TTAAAAATGAAGTTTTAAATCAATCTAAAGTATATATGAGTAAACTTGGT
CTGACACTTACCAATGCTTAATCAGTGAGGCACCTATCTCAGCGATCTGT
CTATTTCGTTCATCCATAGTTGCCTGACTCCCCGTCGTGTAGATAACTAC
GATACGGGAGGGCTTACCATCTGGCCCCAGTGCTGCAATGATACCGCGAG
ACCCACGCTCACCGGCTCCAGATTTATCAGCAATAAACCAGCCAGCCGGA
AGGGCCGAGCGCAGAAGTGGTCCTGCAACTTTATCCGCCTCCATCCAGTC
TATTAATTGTTGCCGGGAAGCTAGAGTAAGTAGTTCGCCAGTTAATAGTT
TGCGCAACGTTGTTGCCATTGCTACAGGCATCGTGGTGTCACGCTCGTCG
TTTGGTATGGCTTCATTCAGCTCCGGTTCCCAACGATCAAGGCGAGTTAC
ATGATCCCCCATGTTGTGCAAAAAAGCGGTTAGCTCCTTCGGTCCTCCGA
TCGTTGTCAGAAGTAAGTTGGCCGCAGTGTTATCACTCATGGTTATGGCA
GCACTGCATAATTCTCTTACTGTCATGCCATCCGTAAGATGCTTTTCTGT
GACTGGTGAGTACTCAACCAAGTCATTCTGAGAATAGTGTATGCGGCGAC
CGAGTTGCTCTTGCCCGGCGTCAATACGGGATAATACCGCGCCACATAGC
AGAACTTTAAAAGTGCTCATCATTGGAAAACGTTCTTCGGGGCGAAAACT
CTCAAGGATCTTACCGCTGTTGAGATCCAGTTCGATGTAACCCACTCGTG
CACCCAACTGATCTTCAGCATCTTTTACTTTCACCAGCGTTTCTGGGTGA
GCAAAAACAGGAAGGCAAAATGCCGCAAAAAAGGGAATAAGGGCGACACG
GAAATGTTGAATACTCATACTCTTCCTTTTTCAATATTATTGAAGCATTT
ATCAGGGTTATTGTCTCATGAGCGGATACATATTTGAATGTATTTAGAAA
AATAAACAAATAGGGGTTCCGCGCACATTTCCCCGAAAAGTGCCACCTGA
CGTCTAAGAAACCATTATTATCATGACATTAACCTATAAAAATAGGCGTA
TCACGAGGCCCTGATGGCTCTTTGCGGCACCCATCGTTCGTAATGTTCCG
TGGCACCGAGGACAACCCTCAAGAGAAAATGTAATCACACTGGCTCACCT
TCGGGTGGGCCTTTCTGCGTTTATAAGGAGACACTTTATGTTTAAGAAGG
TTGGTAAATTCCTTGCGGCTTTGGCAGCCAAGCTAGATCCAGCTTTTTGC
AAAAGCCTAGGCCTCCAAAAAAGCCTCCTCACTACTTCTGGAATAGCTCA
GAGGCCGAGGCGGCCTCGGCCTCTGCATAAATAAAAAAAATTAGTCAGCC
ATGGGGCGGAGAATGGGCGGAACTGGGCGGAGTTAGGGGCGGGATGGGCG
GAGTTAGGGGCGGGACTATGGTTGCTGACTAATTGAGATGCATGCTTTGC
ATACTTCTGCCTGCTGGGGAGCCTGGGGACTTTCCACACCTGGTTGCTGA
CTAATTGAGATGCATGCTTTGCATACTTCTGCCTGCTGGGGAGCCTGGGG
ACTTTCCACACCCTAACTGACACACATTCCACAGCCAAGCTAGCTTGAAT
TAATTCCCGAGCCCTTCCAATACAAAAACTAATTAGACTTTGAGTGATCT
TGAGCCTTTCCTAGTTTTTGTATTGGAAGGGCTCGTCGCCAGTCTCATTG
AGAAGGCATGTGCGGACGATGGCTTCTGTCACTGCAAAGGGGTCACAATT
GGCAGAGGGGCGGCGGTCTTCAAAGTAACCTTTCTTCTCCTGGCCGAGCC
GAGAATGGGAGTAGAGCCGACTGCTTGATTCCCACACCAATCTCCTCGCC
GCTCTCACTTCGCCTCGTTCTCGTGGCTCGTGGCCCTGTCCACCCCGTCC
ATCATCCCGCCGGCCACCGCTCAGAGCACCTTCCACCATGGCCACCTCAG
CAAGTTCCCACTTGAACAAAAACATCAAGCAAATGTACTTGTGCCTGCCC
CAGGGTGAGAAAGTCCAAGCCATGTATATCTGGGTTGATGGTACTGGAGA
AGGACTGCGCTGCAAAACCCGCACCCTGGACTGTGAGCCCAAGTGTGTAG
AAGAGTTACCTGAGTGGAATTTTGATGGCTCTAGTACCTTTCAGTCTGAG
GGCTCCAACAGTGACATGTATCTCAGCCCTGTTGCCATGTTTCGGGACCC
CTTCCGCAGAGATCCCAACAAGCTGGTGTTCTGTGAAGTTTTCAAGTACA
ACCGGAAGCCTGCAGAGACCAATTTAAGGCACTCGTGTAAACGGATAATG
GACATGGTGAGCAACCAGCACCCCTGGTTTGGAATGGAACAGGAGTATAC
TCTGATGGGAACAGATGGGCACCCTTTTGGTTGGCCTTCCAATGGCTTTC
CTGGGCCCCAAGGTCCGTATTACTGTGGTGTGGGCGCAGACAAAGCCTAT
GGCAGGGATATCGTGGAGGCTCACTACCGCGCCTGCTTGTATGCTGGGGT
CAAGATTACAGGAACAAATGCTGAGGTCATGCCTGCCCAGTGGGAGTTCC
AAATAGGACCCTGTGAAGGAATCCGCATGGGAGATCATCTCTGGGTGGCC
CGTTTCATCTTGCATCGAGTATGTGAAGACTTTGGGGTAATAGCAACCTT
TGACCCCAAGCCCATTCCTGGGAACTGGAATGGTGCAGGCTGCCATACCA
ACTTTAGCACCAAGGCCATGCGGGAGGAGAATGGTCTGAAGTAAGTAGCT
TCCTCTGGAGCCATCTTTATTCTCATGGGGTGGAAGGGCTTTGTGTTAGG
GTTGGGAAAGTTGGACTTCTCACAAACTACATGCCATGCTCTTCGTGTTT
GTCATAAGCCTATCGTTTTGTACCCGTTGGAGAAGTGACAGTACTCTAGG
AATAGAATTACAGCTGTGATATGGGAAAGTTGTCACGTAGGTTCAAGCAT
TTAAAGGTCTTTAGTAAGAACTAAATACACATACAAGCAAGTGGGTGACT
TAATTCTTACTGATGGGAAGAGGCCAGTGATGGGGGTCTTCCCATCCAAA
AGATAATTGGTATTACATGTTGAGGACTGGTCTGAAGCACTTGAGACATA
GGTCACAAGGCAGACACAGCCTGCATCAAGTATTTATTGGTTTCTTATGG
AACTCATGCCTGCTCCTGCCCTTGAAGGACAGGTTTCTAGTGACAAGGTC
AGACCCTCACCTTTACTGCTTCCACCAGGCACATCGAGGAGGCCATCGAG
AAACTAAGCAAGCGGCACCGGTACCACATTCGAGCCTACGATCCCAAGGG
GGGCCTGGACAATGCCCGTCGTCTGACTGGGTTCCACGAAACGTCCAACA
TCAACGACTTTTCTGCTGGTGTCGCCAATCGCAGTGCCAGCATCCGCATT
CCCCGGACTGTCGGCCAGGAGAAGAAAGGTTACTTTGAAGACCGCCGCCC
CTCTGCCAATTGTGACCCCTTTGCAGTGACAGAAGCCATCGTCCGCACAT
GCCTTCTCAATGAGACTGGCGACGAGCCCTTCCAATACAAAAACTAATTA
GACTTTGAGTGATCTTGAGCCTTTCCTAGTTCATCCCACCCCGCCCCAGC
TGTCTCATTGTAACTCAAAGGATGGAATATCAAGGTCTTTTTATTCCTCG
TGCCCAGTTAATCTTGCTTTTATTGGTCAGAATAGAGGAGTCAAGTTCTT
AATCCCTATACACCCAACCCTCATTTCTTTTCTATTTAGCTTTCTAGTGG
GGGTGGGAGGGGTAGGGGAAGGGAACGTAACCACTGCTTCATCTCATCAG
GAATGCATGTCCAGTAGGCAGAGCTGCCACAGAGTGGGTGTATTTGTGGA
GGAGGACTTTTTCTTCAGGACAGTTAAAAGAGCAGGTCCACTGCTTGGAT
TGACAATTCCCCTATAGGTAGAGAGCTGCTAGTTCTTCAGGTAAAACCAA
CTTTCTATTCCAAATGGAAGTTAGGTGAGGAGTAGTGGGAGGAGTTCATG
CCCTCCATGAAGACAGCTCAGTGTATCACCTGACAGATGGGTAGCCCTAC
TGTAAAAGAAGGAAAAGTTATTTCTGGGTCCTCCATTTATAACACAAAGC
AGAGTAGTATTTTTATATTTAAATGTAAAAACAAAAGTTATATATATGGA
TATGTGGATATATGTGTATTTCTAATTGAGGAAACCATCCTAGTTACTGG
GTTTGCCAAGTTTGAAGAGCTTGGTTAACAAGAAAGGATCTCTTGAGTAG
AGGTGGGGGTGCAGTACCAGGAAAGGTGGTTATCTGGGGCTCAGCGCTTT
ATTACTATGTGGGGTTTCCCTGCCCACTCTGCAGGAGCAGATGCTGGACA
GGTAGCAGGGTGGGACACCAGTGCTTGCCACCACCTGTCCCTGTGCTTAG
GCTAAGATGCATATGTATCCACACAGAGTTAGCAGGATGGAGTTGGCTGG
TCAACTTGAACATTGTTACTGATAGGGGTGGGTGGGGTTTATTTTTTGGT
GGGACTAGCATGTCACTAAAGCAGGCCTTTTGATATATTAAATTTTTTAA
AGCAAAACAAGTTCAGCTTTTAATCAACTTTGTAGGGTTTCTAACTTTAC
AGAATTGCCTGTTTGTTTCAGTGTCTCCATCCACTTTGCTCTTGGAGGAA
CGGAGGACAGGCAGACCTGGAGTTAAAACATTTGTCATTTTGTGTCATAG
TGTCTACTTTCTCCCAGCAGAATATTCCTTTCCTTCTTAGGAGTCCTATG
GAGTTTTGTTTTTGTTTTTTTTCTATTACGATAAACATACCCCACCTCCA
TTCTGGCTTGCCCTGCTGTTCTCTGGTTGTTTGTGTGCTGTCCGCAGCAG
GCTGCCTGTGGTTTTCTCTTGCCATGACGACTTCTAATTGCCATGTACAG
TATGTTCAGTTAGATAACTCCTCATTGTAAACAGACTGTAACTGCCAGAG
CAGCGCTTATAAATCAACCTAACATTTATAAGATTTCCTCTTGACTTGTT
TCTTTGTGGTTGGGGGAGGAAGAAAAAAAAAAGCGTGCAGTATTTTTTTG
TTCCTTCATTTCCTATCAAAAGAAAGGGGAGTGGTTCTGTTTTGTTTACT
CGCAAAATAAGCTAGCTTATCTATTGGCTTTTCTTTTTTTTTTTTTTTTT
AAACGGGCTTTTTCTTGTACCTATAATTTGGGGTAAGGTGTGAGAGTTTT
TATAGTTTTTTGAGACAGGGTCTTGGTGTATACCCTTGGCTGGCCTGGAG
CTAACTATGTAGACTGGGCTAGCCTTTAACTTGCAGTTCTGCTTTCAATT
AGGGTTTATACATTTAGTCTTGGCAATTCCTAGTTCCACGTTTAATCTCT
TTACATTTCAAAGCAGTGTTATCTGAAGAGTTCAGGCGCAGAGTCAATTC
AATAGAGTTACACAAAAACCTAAAAAACAAGTTTTAAATACCAAGTTATG
TTGGCCTGGCCACTTTTCACAGCTGTCCACAACTCAATGTGACAAGGCTA
CAAATTGGATATACTAGAATTTCCTGGTGATTTGGAACCCCTGCTTCATT
TCCCGGAACCAGGGCTTTTGGTGACAGTCCTAGCTTATCAGATTATTTAA
AACAGTTACTCTTCCTGCCCTTCTTCCTGAGACCTTTGTCCAGCTGCCAT
GAGCCATCTACACAGTACTTGCTTCCCTGTTGAAGTCACTGAAGGCACAT
CAGCCCAAGACATAAAGGCTTGTCCCGGATTCACTAGCCTGGTGAACTTG
TGGTTCTCTGATGTTTTGTCCTGTTTTGTTGTGATTTAGTCTCAAATTTC
CCAGCCTGGTTTGAAAATCTGGGCTCCCAGCCTTCAATAAGGAGGACTAC
AGATATGTACGACTGAGCCTTGATTCCAGCCTCATGTTTATACGTCTGTG
CTCAGCTCCCTGAAGGTTCCAGTTTGAAACTCAATAATCCAGGGGTCAGA
AAGTCTTGATCTTATCCCCACAGTATGGCACCAAGCCTGGCTGAGCCTTC
TGACTTAGTCTGCCCTGTTGCTATTTAAGCACTTTTCTTCACTAGGCTAA
AAATAAAAGGAGCTTCCTCCTTTGCCATGGCGCTGTGCATGATAGGAAAA
GGTAGCTATCTACTAGCATATTAACTCCACTGTTTTTGCTTTGTGTGTTT
GGTTTTTGAGGAAGGGTCTCAACTGTGTATCCCTGGCTGGCCTGGCCGGA
TCTAGCTTCGTGTCAAGGACGGTGACTGCAGTGAATAATAAAATGTGTGT
TTGTCCGAAATACGCGTTTTGAGATTTCTGTCGCCGACTAAATTCATGTC
GCGCGATAGTGGTGTTTATCGCCGATAGAGATGGCGATATTGGAAAAATC
GATATTTGAAAATATGGCATATTGAAAATGTCGCCGATGTGAGTTTCTGT
GTAACTGATATCGCCATTTCCCCAAAAGTGATTTTTGGGCATACGCGATA
TCTGGCGGATAGCGCTTATATCGTTTACGGGGGATGGCGATAGACGACTT
TGGTGACTTGGGCGATTCTGTGTGTCGCAAATATCGCAGTTTCGATATAG
GTGACAGACGATATGAGGCTATATCGCCGATAGAGGCGACATCAAGCTGG
CACATGGCCAATGCATATCGATCTATACATTGAATCAATATTGGCCATTA
GCCATATTATTCATTGGTTATATAGCATAAATCAATATTGGCTATTGGCC
ATTGCATACGTTGTATCCATATCATAATATGTACATTTATATTGGCTCAT
GTCCAACATTACCGCCATGTTGACATTGATTATTGACTAGTTATTAATAG
TAATCAATTACGGGGTCATTAGTTCATAGCCCATATATGGAGTTCCGCGT
TACATAACTTACGGTAAATGGCCCGCCTGGCTGACCGCCCAACGACCCCC
GCCCATTGACGTCAATAATGACGTATGTTCCCATAGTAACGCCAATAGGG
ACTTTCCATTGACGTCAATGGGTGGAGTATTTACGGTAAACTGCCCACTT
GGCAGTACATCAAGTGTATCATATGCCAAGTACGCCCCCTATTGACGTCA
ATGACGGTAAATGGCCCGCCTGGCATTATGCCCAGTACATGACCTTATGG
GACTTTCCTACTTGGCAGTACATCTACGTATTAGTCATCGCTATTACCAT
GGTGATGCGGTTTTGGCAGTACATCAATGGGCGTGGATAGCGGTTTGACT
CACGGGGATTTCCAAGTCTCCACCCCATTGACGTCAATGGGAGTTTGTTT
TGGCACCAAAATCAACGGGACTTTCCAAAATGTCGTAACAACTCCGCCCC
ATTGACGCAAATGGGCGGTAGGCGTGTACGGTGGGAGGTCTATATAAGCA
GAGCTCGTTTAGTGAACCGTCAGATCGCCTGGAGACGCCATCCACGCTGT
TTTGACCTCCATAGAAGACACCGGGACCGATCCAGCCTCCGCGGCCGGGA
ACGGTGCATTGGAACGCGGATTCCCCGTGCCAAGAGTGACGTAAGTACCG
CCTATAGAGTCTATAGGCCCACCCCCTTGGCTTCTTATGCATGCTATACT
GTTTTTGGCTTGGGGTCTATACACCCCCGCTTCCTCATGTTATAGGTGAT
GGTATAGCTTAGCCTATAGGTGTGGGTTATTGACCATTATTGACCACTCC
CCTATTGGTGACGATACTTTCCATTACTAATCCATAACATGGCTCTTTGC
CACAACTCTCTTTATTGGCTATATGCCAATACACTGTCCTTCAGAGACTG
ACACGGACTCTGTATTTTTACAGGATGGGGTCTCATTTATTATTTACAAA
TTCACATATACAACACCACCGTCCCCAGTGCCCGCAGTTTTTATTAAACA
TAACGTGGGATCTCCACGCGAATCTCGGGTACGTGTTCCGGACATGGGCT
CTTCTCCGGTAGCGGCGGAGCTTCTACATCCGAGCCCTGCTCCCATGCCT
CCAGCGACTCATGGTCGCTCGGCAGCTCCTTGCTCCTAACAGTGGAGGCC
AGACTTAGGCACAGCACGATGCCCACCACCACCAGTGTGCCGCACAAGGC
CGTGGCGGTAGGGTATGTGTCTGAAAATGAGCTCGGGGAGCGGGCTTGCA
CCGCTGACGCATTTGGAAGACTTAAGGCAGCGGCAGAAGAAGATGCAGGC
AGCTGAGTTGTTGTGTTCTGATAAGAGTCAGAGGTAACTCCCGTTGCGGT
GCTGTTAACGGTGGAGGGCAGTGTAGTCTGAGCAGTACTCGTTGCTGCCG
CGCGCGCCACCAGACATAATAGCTGACAGACTAACAGACTGTTCCTTTCC
ATGGGTCTTTTCTGCAGTCACCGTCCTTGACACGAAGCTTGCCGCCACCA
TGCCGCTGCTGCTACTGCTGCCCCTGCTGTGGGCAGGGGCCCTGGCTATG
GATCATCACCATCACCATCACCATCACGGTGGCGGTCTGAACGACATCTT
CGAGGCTCAGAAAATCGAATGGCACGAACGGAAAATCCCCAAAGTAGGAC
ATACTTTTTTCCAAAAGCCTGAGAGTTGCCCGCCTGTGCCAGGAGGTAGT
ATGAAACTCGACATTGGCATCATCAATGAAAACCAGCGCGTTTCCATGTC
ACGTAACATCGAGAGCCGCTCCACCTCCCCCTGGAATTACACTGTCACTT
GGGACCCCAACCGGTACCCCTCGGAAGTTGTACAGGCCCAGTGTAGGAAC
TTGGGCTGCATCAATGCTCAAGGAAAGGAAGACATCTCCATGAATTCCGT
TCCCATCCAGCAAGAGACCCTGGTCGTCCGGAGGAAGCACCAAGGCTGCT
CTGTTTCTTTCCAGTTGGAGAAGGTGCTGGTGACTGTTGGCTGCACCTGC
GTCACCCCAGTCATCCACCATGTGCAGTAATGACTCGAGCAATTGGCTAG
AGTCGACGCCCCTCTCCCTCCCCCCCCCCTAACGTTACTGGCCGAAGCCG
CTTGGAATAAGGCCGGTGTGCGTTTGTCTATATGTTATTTTCCACCATAT
TGCCGTCTTTTGGCAATGTGAGGGCCCGGAAACCTGGCCCTGTCTTCTTG
ACGAGCATTCCTAGGGGTCTTTCCCCTCTCGCCAAAGGAATGCAAGGTCT
GTTGAATGTCGTGAAGGAAGCAGTTCCTCTGGAAGCTTCTTGAAGACAAA
CAACGTCTGTAGCGACCCTTTGCAGGCAGCGGAACCCCCCACCTGGCGAC
AGGTGCCTCTGCGGCCAAAAGCCACGTGTATAAGATACACCTGCAAAGGC
GGCACAACCCCAGTGCCACGTTGTGAGTTGGATAGTTGTGGAAAGAGTCA
AATGGCTCTCCTCAAGCGTATTCAACAAGGGGCTGAAGGATGCCCAGAAG
GTACCCCATTGTATGGGATCTGATCTGGGGCCTCGGTGCACATGCTTTAC
ATGTGTTTAGTCGAGGTTAAAAAAACGTCTAGGCCCCCCGAACCACGGGG
ACGTGGTTTTCCTTTGAAAAACACGATGATAATATGGCCATGGTGAGCAA
GGGCGAGGAGCTGTTCACCGGGGTGGTGCCCATCCTGGTCGAGCTGGACG
CCGACGTAAACGCCCACAAGTTCAGCGTGTCCGGCGAGGGCGAGGGCGAT
GCCACCTACGGCAAGCTGACCCTGAAGTTCATCTGCACCACCGGCAAGCT
GCCCGTGCCCTGGCCCACCCTCGTGACCACCCTGACCTACGGCGTGCAGT
GCTTCAGCCGCTACCCCGACCACATGAAGCAGCACGACTTCTTCAAGTCC
GCCATGCCCGAAGGCTACGTCCAGGAGCGCACCATCTTCTTCAAGGACGA
CGGCAACTACAAGACCCGCGCCGAGGTGAAGTTCGAGGGCGACACCCTGG
TGAACCGCATCGAGCTGAAGGGCATCGACTTCAAGGAGGACGGCAACATC
CTGGGGCACAAGCTGGAGTACAACTACAACAGCCACAACGTCTATATCAT
GGCCGACAAGCAGAAGAACGGCATCAAGGTGAACTTCAAGATCCGCCACA
ACATCGAGGACGGCAGCGTGCAGCTCGCCGACCACTACCAGCAGAACACC
CCCATCGGCGACGGCCCCGTGCTGCTGCCCGACAACCACTACCTGAGCAC
CCAGTCCGCCCTGAGCAAAGACCCCAACGAGAAGCGCGATCACATGGTCC
TGCTGGAGTTCGTGACCGCCGCCGGGATCACTCTCGGCATGGACGAGCTG
TACAAGCAAAATCACTAGT

### Example 3

### Generation of the A6 VL Expression Vector

In order to generate a control protein of a similar molecular weight as the IL-17 cytokine, the light chain (variable region together with its constant region) of NI-0501 monoclonal antibody (an anti-IFNγ monoclonal antibody described in PCT Publication No. WO 06/109191) was sub-cloned in an expression vector under the control of the hCMV promoter. GFP was cloned downstream of the A6 VL cDNA as a second cistron under the control of the same CMV promoter. The two cistrons (A6 VL and GFP) were separated by viral internal ribosome entry site (IRES) to allow for translation of the second (GFP) cistron. The vector also contained the GS gene under the control of the SV40 promoter for selection of transfected cells in glutamine-free medium using MSX.

### Example 4

### Transfection of NI-0701 Vector Versus Native Human IL-17F

The CHOK1SV cell line, property of Lonza Biologics, plc, was used to generate either, pools through semi-stable transfection or, cell lines through stable transfection for the production of human IL-17F and NI-0701. The word “transfection” used herein describes the introduction of linearized DNA into cells by electroporation. The expression “semi-stable transfection” means the generation, under selection pressure, of recombinant protein-expressing pools, i.e. mixtures of cell lines. The expression “stable transfection” means the generation, under selective pressure, of isolated recombinant protein-producing cell lines.

Briefly, exponentially growing cells in the medium CD-CHO (Invitrogen) supplemented with 6 mM of L-glutamine, were electroporated under the following conditions: in a 0.4 cm cuvette, 1.0×10⁷ viable cells in 700 μL of fresh CD-CHO were gently mixed with 40 μg of DNA in 100 μL of Tris EDTA buffer solution, pH 7.4, immediately followed by delivering of a single pulse of 300 volts, 900 μF.

For each DNA construction, the contents of 4 cuvettes were immediately transferred in 200 mL of fresh pre-warmed CD-CHO. This cell suspension was subsequently distributed in three tissue culture-treated T75 flasks to generate three 50 mL semi-stable pools; the remaining 50 mL of cell suspension was used to generate stable cell lines by limiting dilution in ten 96-well plates (50 μL per well). Afterwards, the T75 flasks and 96-well plates were placed in a humidified incubator set at 10% CO₂ in air and a temperature of 37° C.

Approximately twenty-four hours after transfection, selective pressure (by MSX supplementation at 50 μM) was applied to both stable and semi-stable transfections: in the T75 flasks, 25 μL of a 100 mM stock solution of MSX in PBS were added whilst in the 96-well plates, 150 μL of pre-warmed CD-CHO supplemented with 66.6 μM of MSX was dispensed per well. Finally, plates and flasks were rapidly placed back to the incubator.

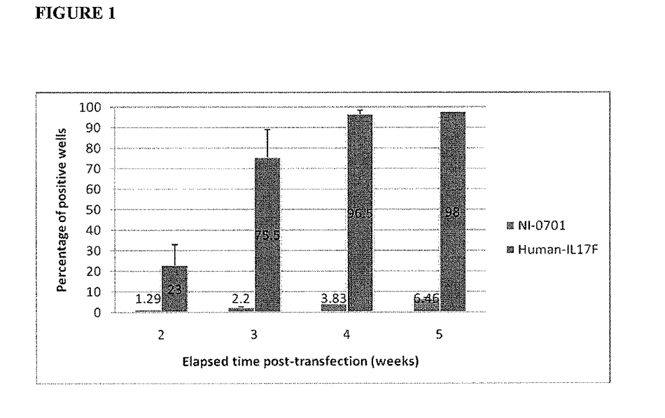

In the stable transfection plates, the emergence of cell lines was assessed by frequent visual observations with the aid of a mirror to conveniently display the bottom of the plates. A “positive well” is defined as a well presenting one or more transfectant colony. FIG. 1 shows that well plates seeded with cells stably transfected with human IL-17F have consistently higher percentages of positive wells representing one or more transfectant colonies beginning at 2 weeks and continuing to 5 weeks post-transfection. The success of IL-17F transfected cells is demonstrates approximately a 16-fold improvement over control, NI-0701-transfected, cells.

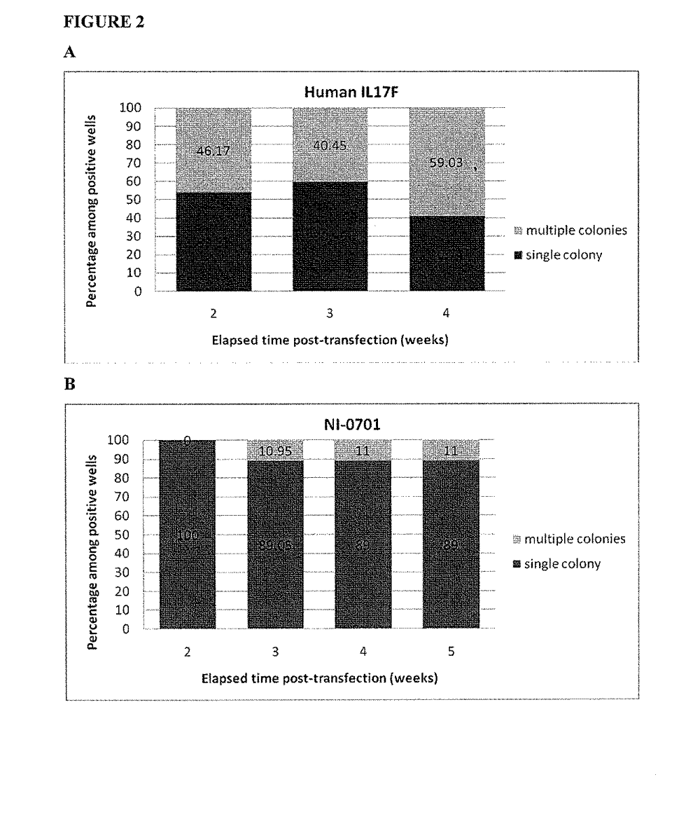

FIG. 2 shows that well plates seeded with IL-17F-transfected cells contain a greater proportion of multiple colonies per well than single colonies per well when compared to NI-0701-transfected cells. Percentage values represent the averages of 2 independent experiments. A “multiple colonies” well is defined as a positive well presenting a number of colonies equal or greater than 2; in contrast, a “single colony” well consists of a positive well showing one isolated transfectant colony.

The data of FIGS. 1 and 2, taken together, show that IL-17F-mediated transfections are more efficacious than NI-0701-mediated stable transfections. The expression of IL-17F greatly increases both the speed of appearance and number of transfected cells resistant to selective pressure.

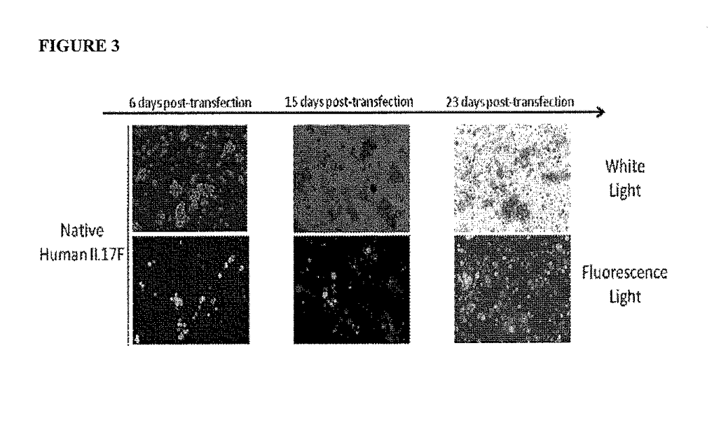

In the semi-stable pools, cell growth and GFP transgene expression were periodically observed by visual examination under fluorescence microscope. The majority of cells resistant to selective pressure are positive for GFP expression at 6, 15, and 23 days post transfection when compared to brightfield illumination, suggesting that the resistant cell lines are expressing human IL-17F (FIG. 3).

### Example 5

### Evaluating the Effects of Exogenous Human IL-17F on Transfection Efficacy

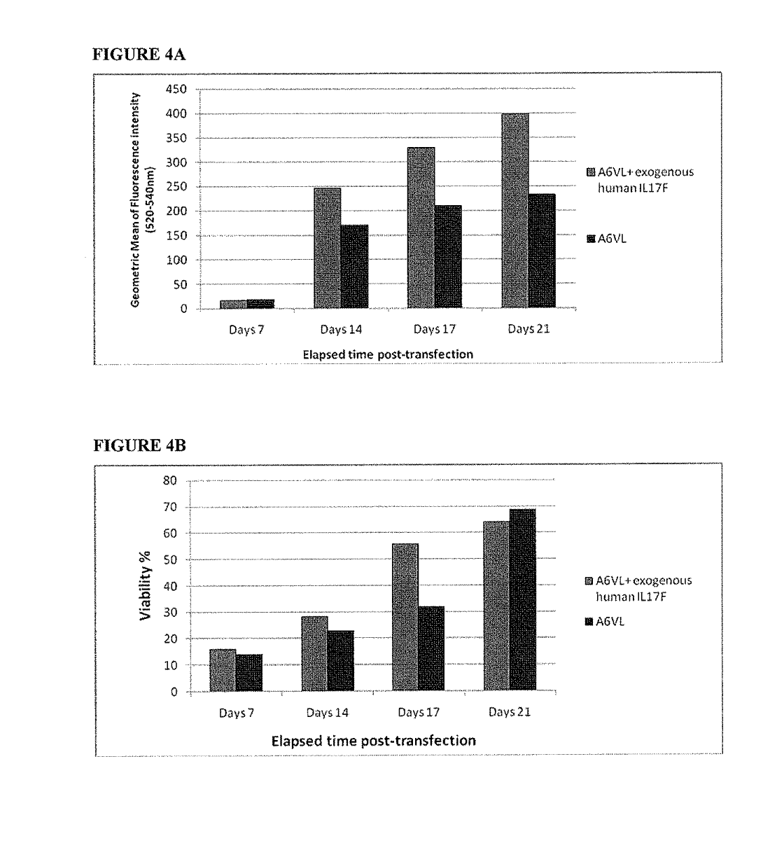

Semi-stable transfections of the A6 VL construct (A6VL-IRES-GFP) into CHOK1SV cells were performed in the presence of culture media either containing or lacking exogenous recombinant human IL-17F. At days 7, 14, 17 and 21, semi-stable pools were analyzed for the expression of GFP by FACS analysis. The overall viability of the cells within the semi-stable pools was determined using an automatic cell counter following trypan blue staining. The data show that at days 14, 17, and 21, cells that were exposed to IL-17F expressed higher levels of GFP than those cells that lacked IL-17F exposure (FIG. 4A). Moreover, the addition of IL-17F increased overall cell viability, especially at day 17 (FIG. 4B).

### Example 6

### Transfection of Other Members of the IL-17 Cytokine Family

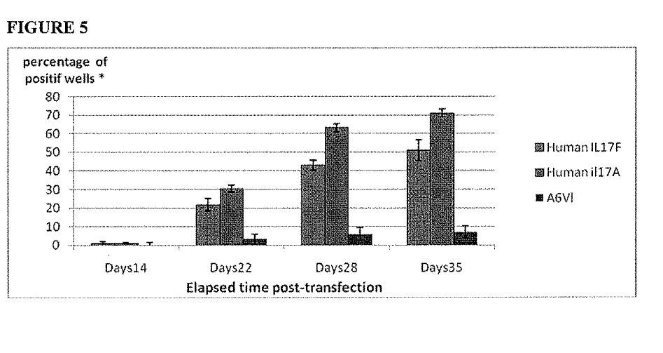

The IL-17 family includes, but is not limited to, IL-17A, IL-17B, IL-17C, IL-17D, IL-17E, and IL-17F. The first IL-17 family member to be evaluated is IL-17A, because IL-17F and IL-17A share the highest degree of amino acid sequence homology and identity. A6VL, IL-17F and IL-17A constructs were stably transfected in CHOK1SV cells. Transfected cells were assessed for the number of positive wells 14, 22, 28 and 35 days post transfection. FIG. 5 shows that at days 22, 28 and 35, the average number of positive wells per 96-well plate is higher for IL-17A- or IL-17F-transfected cells than for the A6VL-transfected cells. Thus, both IL-17A and IL-17F decreased the time of appearance (or increased appearance speed) and increased the number of positive wells.

### Example 7

### Comparison Between Human and Rat IL-17F

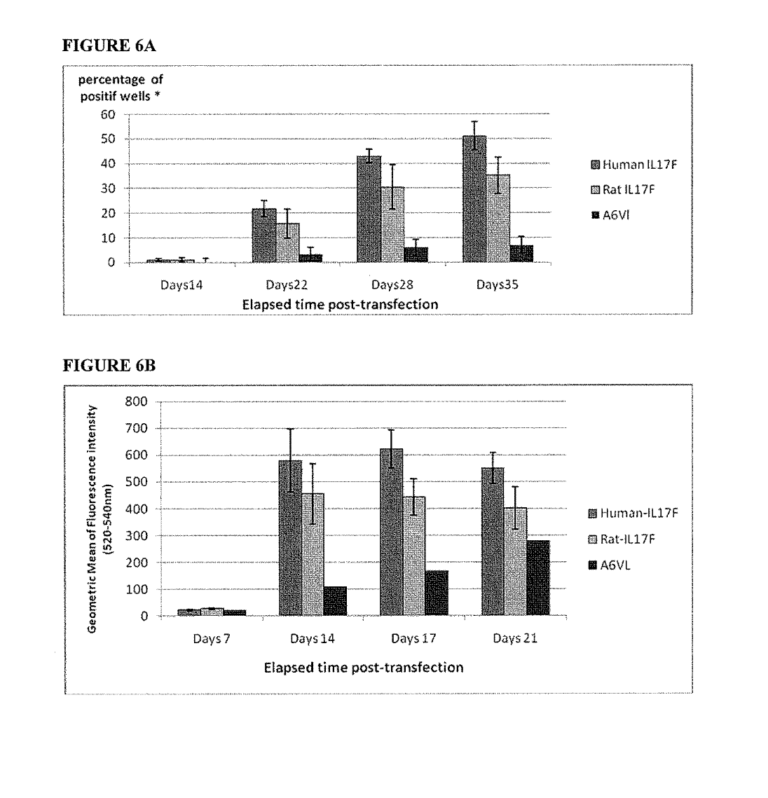

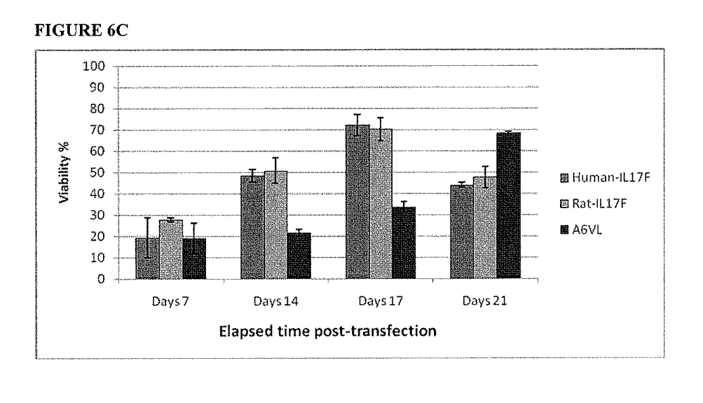

Rat IL-17F has a high sequence homology with its human homologue. Therefore, the individual effects of rat and human IL-17F on transfection ability were determined. Human IL-17F, rat IL-17F and A6VL constructs were either stably or semi-stably transfected into CHOK1SV cells. For stable transfections, the number of positive wells was assessed 14, 22, 28 and 35 days post transfection. Semi-stable pools were analyzed for the expression of GFP by FACS analysis. The overall viability of the cells within the semi-stable pools was determined using an automatic cell counter following trypan blue staining. FIG. 6A shows that at days 22, 28 and 35, the average number of positive wells per 96-well plate is higher for human IL-17F- and rat IL-17F-transfected cells than for the A6VL-transfected cells. FIG. 6B shows that at days 14, 17, and 21, cells transfected with human or rat IL-17F expressed higher levels of GFP than those cells transfected with A6VL. Moreover, cells transfected with human IL-17F or rat IL-17F have a higher viability at days 14 and 17 than cells transfected with A6VL at the same date (FIG. 6C). Thus, transfection with both human and rat IL-17F decreased the time of appearance (or increased appearance speed) and increased the number of positive wells in stable transfections. Furthermore, transfection with both human and rat IL-17F increased the level of recombinant protein expression as assessed by the level of GFP expression.

### Example 8

### Transfection of IL-17F in Other CHO Cell Lines

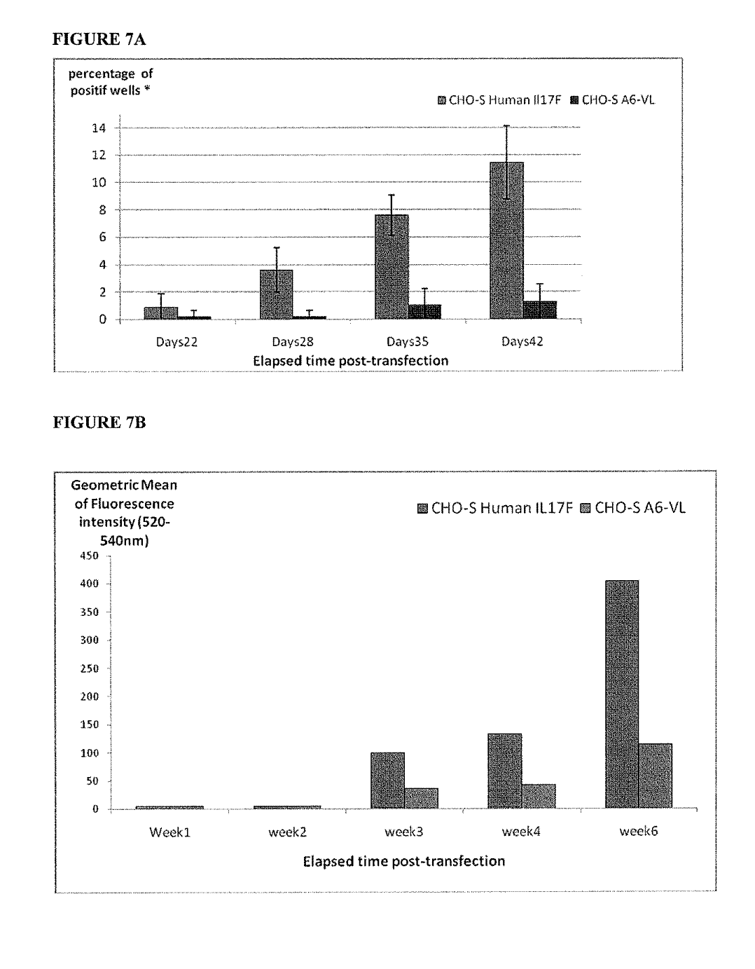

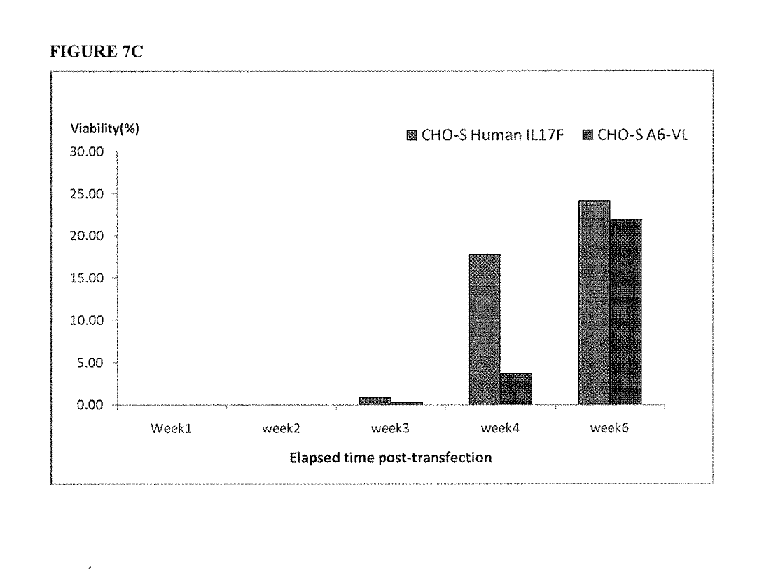

To determine if the effect seen on CHOK1SV cells was not unique to this cell line, the original experiment was reproduced using the CHO—S cell line (Invitrogen). Human IL-17F and A6VL constructs were either stably or semi-stably transfected into CHO—S cells. For stable transfections, the number of positive wells was assessed 22, 28 35, and 42 days post transfection. Semi-stable pools were analyzed for the expression of GFP by FACS analysis at weeks 1, 2, 3, 4 and 6. The overall viability of the cells within the semi-stable pools was determined using an automatic cell counter following trypan blue staining. FIG. 7A shows that the number of positive wells increased by a factor of 5 for cells transfected with IL-17F compared to cells transfected with A6VL. With respect to semi-stable transfections, FIG. 7B shows that the average GFP expression level at weeks 3, 4 and 6 increased by a factor of 4 for cells transfected with IL-17F compared to cells transfected with A6VL. Moreover, the viability of cells transfected with IL-17F significantly increased at week 4 compared to cells transfected with A6VL. Thus, IL-17F had a similar effect on CHO—S and CHOK1SV cells.

### Example 9

### Stable Transfection of CHO Cells with IL-17 IRES GFP Variants Using an Expression Vector System Based on Puromycin Selection

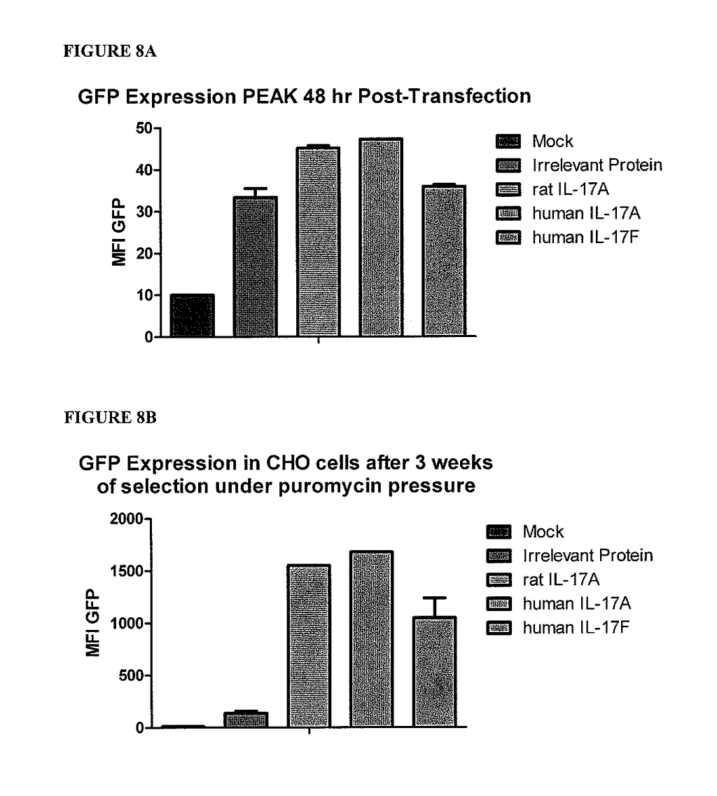

Human Rantes, rat IL-17A, human IL-17A and human IL-17F were subcloned into an expression vector under the control of the EF1-alpha promoter. GFP was subcloned downstream of the Human Rantes, rat IL-17A, human IL-17A and human IL-17F sequences as a second cistron under the control of the same EF1-alpha promoter. The two cistrons were separated by viral internal ribosome entry site (IRES) to allow for translation of the second (GFP) cistron. The vector also contained the puromycin resistance gene. CHO cells or PEAK cells were plated at a density of 4.0×10^{$} cells/well in 6 well culture dishes overnight at 37° C. The following day, 2 μg of DNA were transfected per well using the TransIT-LT1 transfection reagent from Mirius bio following the manufacturer's guidelines. Twenty-four hours post-transfection, PEAK cells were analyzed for GFP expression by flow cytometry (FACS) as a quality control for the DNA/Mirius complexes (FIG. 8A). The GFP expression of each construct was confirmed in this experiment.

In parallel, CHO-transfected cells were placed in static culture under puromycin selection (10 μg/mL). Fresh medium was supplemented as required and clone appearance was monitored by visual inspection. Throughout the duration of the experiment, no difference in either the rate of clone appearance or clone growth was observed for the different expression vectors tested. At 3 weeks post-transfection, clones were pooled and cells analyzed by flow cytometry (FACS) as shown in FIG. 8B. The expression of IL-17 (either human or rat, and either the A or the F isoform) has a striking influence on expression levels of GFP.

### Example 10

### The 15C1 MAb Double Gene Expression Vector

The 15C1 expression vector is a “double gene” vector containing the heavy and light chain variable regions of antibody 15C1 in fusion with the human IgG1 and human kappa constant region cassettes, respectively. The expression of each antibody chain is driven by the strong hCMV promoter. The 15C1 vector also contains the Glutamine Synthetase (GS) gene under the control of the SV40 promoter. GS catalyses synthesis of the essential amino-acid glutamine from glutamic acid, ammonia and ATP. Selection stringency is therefore applied in the absence of glutamine, and eventually in the presence of a specific GS inhibitor, methionine sulphoximine (MSX) for cell lines presenting endogenous GS activity, e.g. CHOK1SV.

The Variable light chain sequence of murine 15C1 antibody, is encoded by the following nucleic acid sequence, NCBI Accession No. CS645163 and SEQ ID NO: 22:

| | |
|---|---|
| 1 | gacattgtga tgacccagtc tccagccacc ctgtctgtga ctccaggtga tagagtctct |
| 61 | ctttcctgca gggccagcca gagtatcagc gaccacttac actggtatca acaaaaatca |
| 121 | catgagtctc cacggcttct catcaaatat gcttcccatg ccatttctgg gatcccctcc |
| 181 | aggttcagtg gcagtggatc agggacagat ttcactctca gcatcaaaag tgtggaacct |
| 241 | gaagatattg gggtgtatta ctgtcaaaat ggtcacagtt ttccgctcac gttcggtgct |
| 301 | gggaccaagc tggagctgaa a |

The Variable heavy chain sequence of murine 15C1 antibody, is encoded by the following nucleic acid sequence, NCBI Accession No. CS645158 and SEQ ID NO: 23:

| | |
|---|---|
| 1 | gatgtgcagc ttcaggagtc aggacctgac ctaatacaac cttctcagtc actttcactc |
| 61 | acctgcactg tcactggcta ctccatcacc ggtggttata gctggcactg gatccggcag |
| 121 | tttccaggaa acaaactgga atggatgggc tacatccact acagtggtta cactgacttc |
| 181 | aacccctctc tcaaaactcg aatctctatc actcgagaca catccaagaa ccagttcttc |
| 241 | ctgcagttga attctgtgac tactgaagac acagccacat attactgtgc aagaaaagat |
| 301 | ccgtccgacg gatttcctta ctggggccaa gggactctgg tcactgtctc tgca |

### Example 11

### Co-Transfection of 15C1 Double Gene Expression Vector and the Human IL-17F Expression Vector

To determine the effect of IL-17F on the level of IgG expression, co-transfection of 15C1 MAb Double Gene Expression Vector together with the human IL-17F Expression Vector was performed.

Human IL-17F and 15C1 constructs were co-transfected by electroporation. Cells were either plated into 96 well plates in order to obtain stable clones or kept as a polyclonal pool of cells in T75 flasks. As a reference standard, the 15C1 MAb double gene expression vector was also transfected alone and the resulting transfected cells were processed in the same way.

For transfected CHO cells plated in 96 well plates, the number of wells in which the presence of a single growing colony could be visible at 22 and 28 days post transfection was evaluated. For each transfection conditions, the supernatant of 20 colonies presenting a similar size and healthy appearance was taken and its human IgG/K concentration determined by Enzyme Linked ImmunoSorbent Analysis (ELISA). For transfected pools, the concentration of human IgG/K was also determined by ELISA at days 7, 14, 21 and 28 post transfection. Briefly, the concentration of 15C1 antibody was evaluated by ELISA using a Goat anti-human IgG Fcγ specific polyclonal antibody (Jackson immunoresearch, 109-005-098) for capture of the whole human IgG/K present in the supernatant and a HRP conjugated-Goat anti-human κ light Chain polyclonal antibody (Sigma, A-7164) for detection.

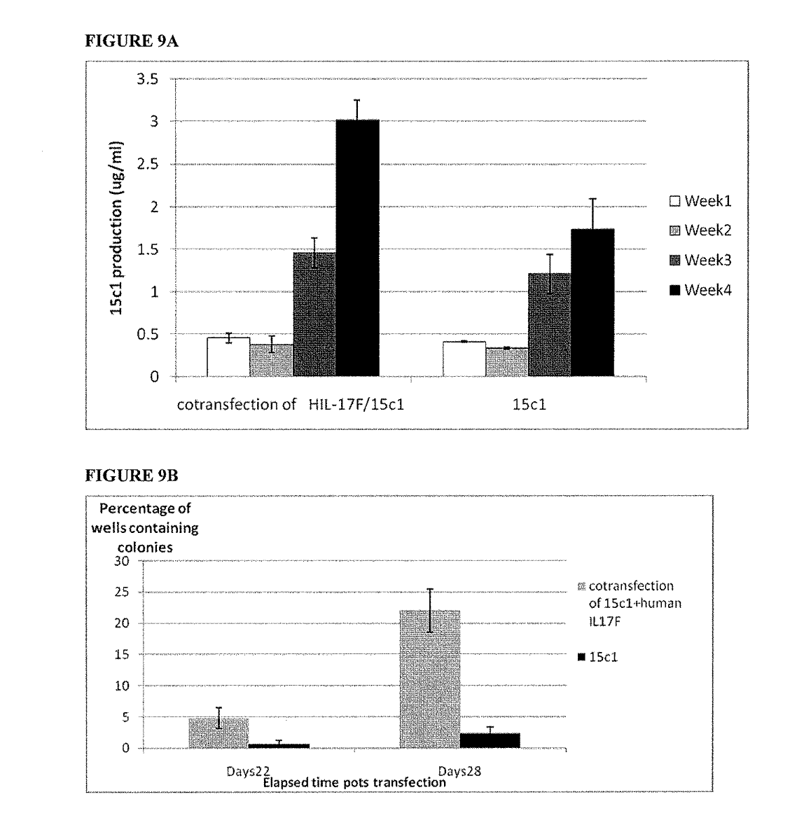

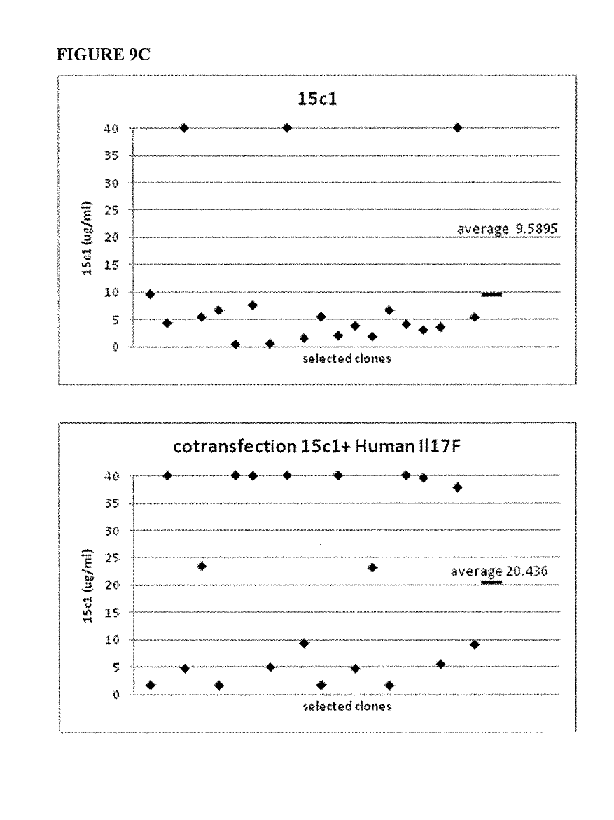

FIG. 9A shows the 15C1 human IgG1/Kappa concentration in the supernatant from transfected pools at 1, 2, 3, or 4 weeks post-transfection. The data show that at 4 weeks post transfection, the concentration of the 15C1 MAb is higher by a factor of 2 in the co-transfection condition compared to the transfection of 15C1 alone. Thus, the data show that IL-17F had a positive effect on 15C1 production.

FIG. 9B shows the number of wells containing 1 or more colonies per 96 well plate at 22 and 28 days post co-transfection (15C1 MAb and human IL-17F) or single transfection (15C1 MAb) The data show that the number of clones obtained in the co-transfection condition increased by factors of 5 and 10 compared to the single transfection condition 22 and 28 days post transfection, respectively.

FIG. 9C shows the level of expression of 15C1 MAb in the supernatant of each 20 individual clones. The data show that the number of high producer clones (those out of range signal in the ELISA) is higher by a factor of 2.5 for the co-transfection condition compared to the 15C1 alone condition. Moreover, the average antibody titer for all 20 clones is higher by a factor 2 in the co-transfection condition compared to 15C1 alone. In the co-transfection condition, all of the 20 clones expressed GFP at a variable but strong level as estimated by fluorescence microscopy. The presence of GFP staining is an accurate indicator for the strong expression of human IL-17F by all the clones because the IL-17F vector contains an IRES-GFP sequence downstream of IL-17F for bicistronic expression (see Example 2).

### Example 12

### Presence of IL-17F Results in More Robust Sub-Cloning of Cells

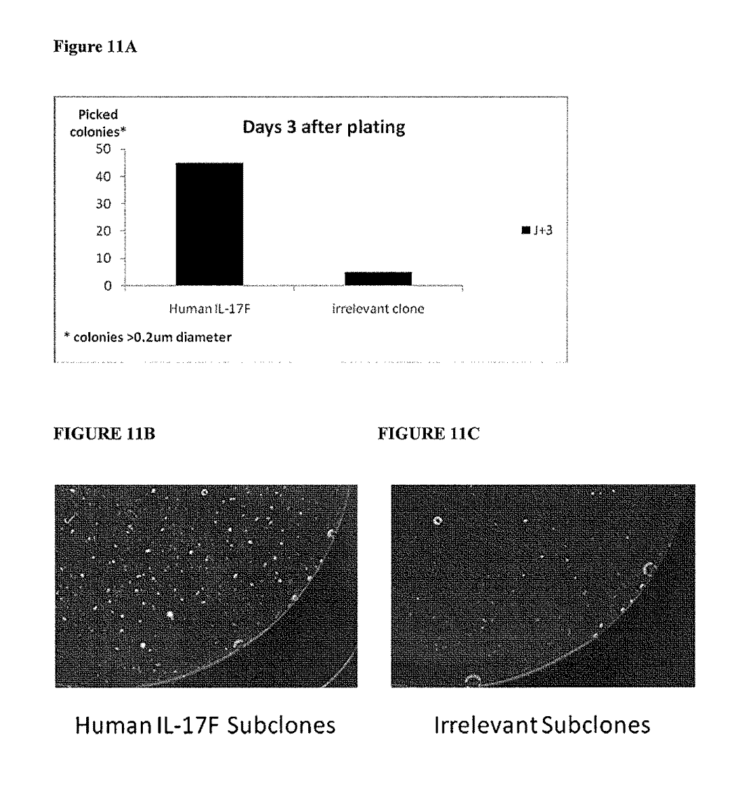

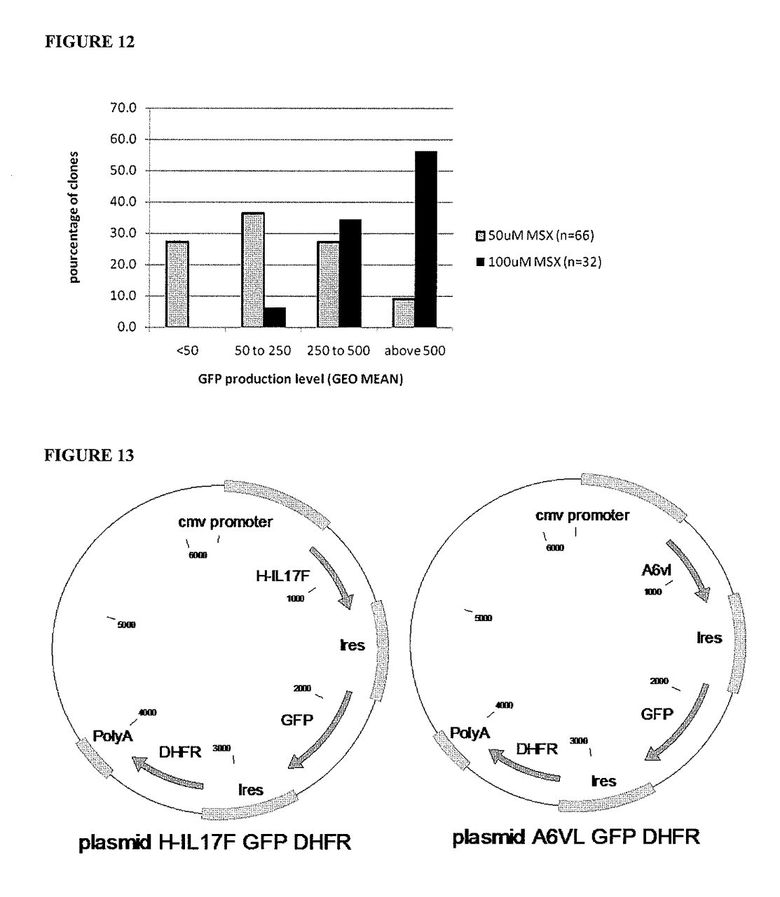

As shown in FIGS. 11A-12, the presence of IL-17F makes the process of sub-cloning of cells more robust. In FIGS. 11A-11C, cells from two CHOK1SV cell lines, 8E11, which expresses IL-17F-IRES-GFP, and C6C5, which expresses an irrelevant MAb, were plated in semi solid medium in a 6 well plate (cellulose acetate containing OptiCHO and conditioned CHO supernatant). Colonies >0.2 um in diameter were picked 3 days post-plating, and isolated clones were analyzed using the ClonePixFL and quantified.

### Example 13

### Presence of IL-17F Allows for Greater Selective Pressure on Transfected Cells and Consequently Higher Resulting Transgene Productivity

In FIG. 12, cells were transfected with an IL-17F IRES GFP expression cassette and plated in 96 well plates under 50 μM or 100 μM MSX selection pressure as indicated. Clones appeared at 3-5 weeks post-transfection and were subsequently analyzed for GFP expression by FACS analysis.

### Example 14

### Stable Transfection of CHODG44 with IL-17F IRES GFP Using an Expression System Based on DHFR Selection

Two cistrons comprising by the Human IL-17F gene or an irrelevant protein and the GFP gene were subcloned into an expression vector under control of the hCMV promoter. These two cistrons were separated by a viral internal ribosome entry site (IRES). The vector (Invitrogen pOptiVEC) also contained, downstream the cloning site, an IRES sequence followed by DHFR gene. This construction therefore allows expression of IL-17F, GFP and the selection marker (DHFR) from a tricistronic mRNA. (FIG. 13)

DHFR (dihydrofolate reductase) catalyzes the reduction of 5,6-dihydrofolate to 5,6,7,8 tetrahydrofolate which is essential for DNA synthesis. The CHODG44 cell line lacks DHFR activity and must be cultivated in a medium supplemented with the purine precursors hypoxanthine and thymidine (HT). Methotrexate (MTX) is a folic acid antagonist which inhibits DHFR activity. As a selection condition, medium without HT and supplemented with MTX was used. CHODG44 cells were transfected using a standard electroporation protocol in medium with HT (00124/00125). Forty-eight hours post transfection, the culture medium was replaced by a medium without HT and with 500 or 1000 nM of MTX. Transfected cells were diluted by 4-fold and plated in 96 wells plate. The rate of clone appearance was evaluated by visual observation, and GFP expression of isolated clones was evaluated by FACS analysis.

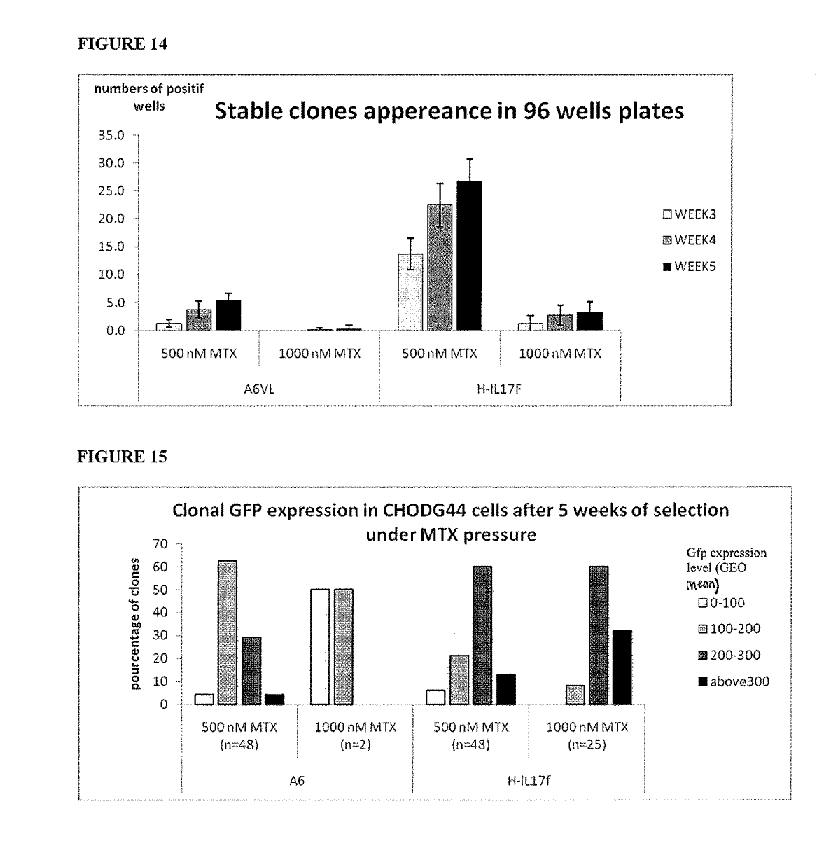

FIG. 14 shows the number of wells containing 1 or more colony per 96 well plates at weeks 3, 4 and 5 post transfection. The data shows that human IL-17F enhances the number of wells presenting one or more transfectant colony by a factor of 5 compared to the control. It also shows that IL-17F is permissive for selection at higher level of selecting agent (2 fold).

FIG. 15 shows the level of GFP expression of individual clones at weeks 5 post transfection. The data demonstrates at 500 nM MTX higher percentage of high and very high GFP producer clones. By raising the selection pressure (500 nM to 1000 nM of MTX), IL-17F allows the reduction of the proportion of lower GFP producer clones and enhances the proportion of very high GFP producer clones.

### Example 15

### Co-Expression of IL-17F and Full IgG in CHO Cells

Plasmids containing simultaneous bi-cistronic expression cassettes were created. The first expression cassette was composed of a double cistronic gene with an IgG light chain sequence followed by IRES and then the GFP gene. The second expression cassette was composed of an IgG heavy chain sequence followed by an IRES then either the Human IL-17F gene or a non-relevant protein gene. These constructions allows for the production of a assembled IgG protein, the GFP and either the human IL-17F or the irrelevant protein in a single plasmid. These “double double gene” vectors were transfected into CHOK1SV using a standard electroporation protocol.

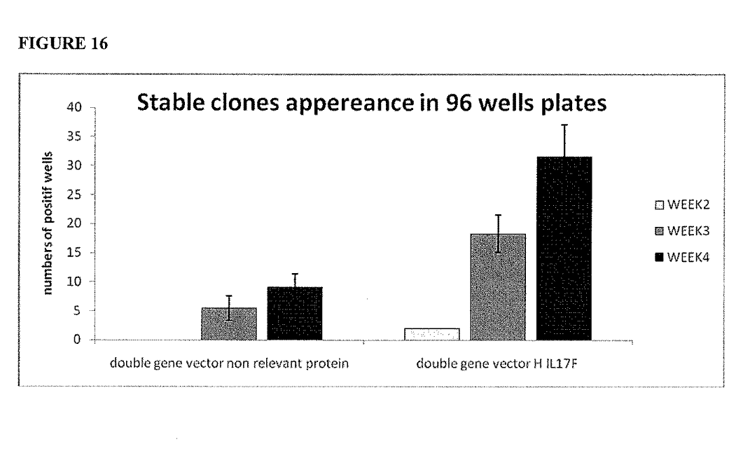

FIG. 16 shows the numbers of wells containing 1 or more colonies per 96 well plates at weeks 3, 4 and 5 post transfection. The data demonstrates that IL-17F enhances clonal appearance.

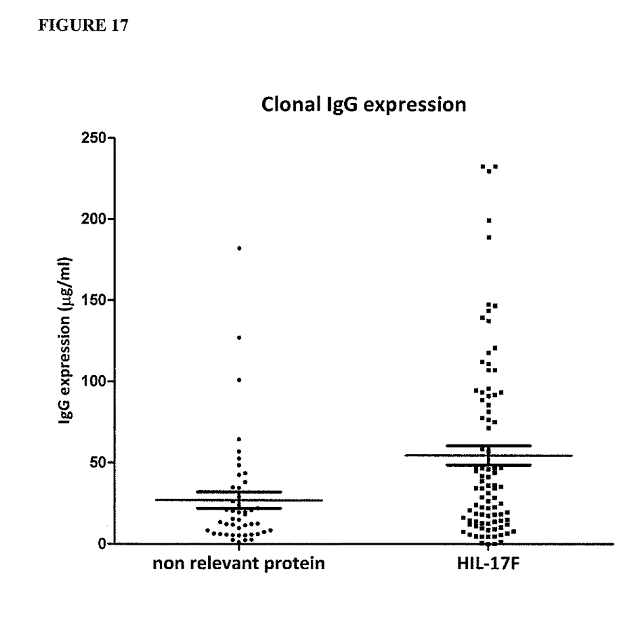

FIG. 17 shows the average level of IgG of individual clones at 4 weeks post-transfection. This data shows that expressing human IL-17F with full IgG protein enhanced the selection of high IgG producer clones.

### Example 16

### Effect of IL-17F on Clonal Selection Using ClonePix^{FL} Technology

CHO cell lines that stably express different levels of human IL-17F protein (arbitrarily called “high”, “medium” and “low” corresponding to their approximate IL-17F expression level) were selected. 6 well plates containing semi-solid medium (with/without conditioned medium) were inoculated with different concentrations of cells (50, 500, 5000 cells/mL). The cells were cultivated for 9 days. The number of single growing colonies was evaluated at day 5 and day 9 under a white light microscope with a ClonePix^{FL} imaging station.

The total number of growing clones from 3 wells inoculated with 3 concentrations of IL-17F expressing CHO cells were determined. The data demonstrated that IL-17F enhanced the number of single growing clones in a medium supplemented or not in conditioned medium. The effect of IL-17F on the number of clones was dose-dependent.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of using IL-17 to enhance a property of modification of a cell with a nucleic acid, the method comprising the step of contacting the cell with said IL-17.

2. The method of claim 1, wherein the exposure to IL-17 causes enhanced expression of the nucleic acid compared to a cell not contacted by IL-17.

3. The method of claim 1, wherein said IL-17 contacts a cell at a time selected from prior to said modification, during said modification, following said modification and combinations thereof.

4. The method of claim 1, wherein said IL-17 contacts a cell continuously.

5. The method of claim 1, wherein said IL-17 contacts a cell by being present in the culture medium.

6. The method of claim 1, wherein IL-17 is produced by a cell transformed to express IL-17.

7. The method of claim 1, wherein said nucleic acid comprises one or more sequences encoding an IL-17 cytokine.

8. The method of claim 7, wherein said IL-17 is IL-17A, IL-17B, IL-17C, IL-17D, IL-17E, or IL-17F.

9. The method of claim 7, wherein said IL-17 is IL-17F.

10. The method of claim 1, wherein said cell is under selective pressure.

11. The method of claim 10, wherein said modification is semi-stable or stable.

12. The method of claim 6, wherein said IL-17 is produced simultaneously or sequentially with said nucleic acid.

13. The method of claim 6, wherein said IL-17 is under the control of an inducible promoter.

14. The method of claim 1, wherein said cell or cell line(s) comprise mammalian cells.

15. The method of claim 1, wherein said cell or cell line(s) comprise human cells.

16. The method of claim 1, wherein said cell or cell line(s) comprise primary cells in culture.

17. The method of claim 1, wherein said cell or cell line(s) comprise hybridoma cells in culture.

18. The method of claim 1, wherein said cell or cell line(s) is a CHO cell, a CHO cell line, or derived from a CHO cell or CHO cell line.

19. The method of claim 1, wherein said enhanced property of modification is selected from increased efficiency, increased selection rate, increased cell growth, increased appearance speed of selected cells, increased number of selected cell lines, increased doubling time of selected cells, increased cell viability, increased cell line stability, reduced sensitivity to medium depletion and combinations thereof.

20. The method of claim 1, wherein said enhanced expression of one or more exogenous gene(s) is increased specific production rate of monoclonal antibody (MAb), increased MAb titer, increased product quality, correlation of IL-17 expression with MAb titer, increased expression following transient modification of transfection-resistant cell-lines, or increased transgene productivity, increased incorporation of exogenous DNA into genomic sequence, increased retention of exogenous DNA, increased uptake of DNA, or increased expression of exogenous DNA.

21. A method of enhancing the efficacy of cell modification, comprising the steps of:
(a) culturing one or more cells or cell line(s) in medium;
(b) contacting one or more cells or cell line(s) with a nucleic acid;
(c) culturing modified cells in medium to express the polypeptide encoded by the nucleic acid wherein cells are exposed to IL-17 prior to or during said contacting step;
wherein one or more cell lines expressing one or more polypeptides is generated that demonstrates an enhanced property of transfection.

22. The method of claim 21, wherein the exposure to IL-17 causes enhanced expression of the nucleic acid compared to a cell not contacted by IL-17.

23. The method of claim 21, wherein said IL-17 contacts a cell at a time selected from prior to said modification, during said modification, following said modification and combinations thereof.

24. The method of claim 21, wherein said IL-17 contacts a cell continuously.

25. The method of claim 21, wherein said IL-17 contacts a cell by being present in the culture medium.

26. The method of claim 21, wherein IL-17 is produced by a cell transformed to express IL-17.

27. The method of claim 21, wherein said nucleic acid comprises one or more sequences encoding an IL-17 cytokine.

28. The method of claim 27, wherein said IL-17 is IL-17A, IL-17B, IL-17C, IL-17D, IL-17E, or IL-17F.

29. The method of claim 27, wherein said IL-17 is IL-17F.

30. The method of claim 21, wherein said a cell is under selective pressure.

31. The method of claim 30, wherein said modification is semi-stable or stable.

32. The method of claim 21, wherein said modification is transient.

33. The method of claim 26, wherein said IL-17 is produced simultaneously or sequentially with said nucleic acid.

34. The method of claim 21, wherein said cell or cell line(s) comprise mammalian cells.

35. The method of claim 21, wherein said cell or cell line(s) comprise human cells.

36. The method of claim 21, wherein said cell or cell line(s) comprise primary cells in culture.

37. The method of claim 21, wherein said cell or cell line(s) comprise hybridoma cells in culture.

38. The method of claim 21, wherein said cell or cell line(s) is a CHO cell, a CHO cell line, or derived from a CHO cell or CHO cell line.

39. The method of claim 21, wherein said enhanced property of modification is selected from increased efficiency, increased selection rate, increased cell growth, increased appearance speed of selected cells, increased number of selected cell lines, increased doubling time of selected cells, increased cell viability, increased cell line stability, reduced sensitivity to medium depletion and combinations thereof.

40. The method of claim 21, wherein said enhanced expression of one or more exogenous gene(s) is increased specific production rate of monoclonal antibody (MAb), increased MAb titer, increased product quality, correlation of IL-17 expression with MAb titer, increased expression following transient modification of transfection-resistant cell-lines, or increased transgene productivity, increased incorporation of exogenous DNA into genomic sequence, increased retention of exogenous DNA, increased uptake of DNA, or increased expression of exogenous DNA.

41. A method of enhancing a property of subcloning or single cell cloning, said method comprising the steps of:
(a) culturing one or more cloned cells or cell line(s) in medium, and
(b) contacting the one or more cloned cells or cell line(s) with an IL-17 composition,
wherein the one or more cloned cells or cell lines exhibit an enhanced property after contact with the IL-17 composition.

42. The method of claim 41, wherein said IL-17 composition contacts a cell continuously.

43. The method of claim 41, wherein said IL-17 contacts a cell by being present in the culture medium.

44. The method of claim 41, wherein IL-17 is produced by a cell transformed to express IL-17.

45. The method of claim 41, wherein said IL-17 comprises an IL-17 cytokine selected from IL-17A, IL-17B, IL-17C, IL-17D, IL-17E, and IL-17F.

46. The method of claim 41, wherein said IL-17 composition comprises IL-17F.

47. The method of claim 41, wherein said a cell is under selective pressure.

48. The method of claim 41, wherein said cloned cell or cell line(s) comprise mammalian cells.

49. The method of claim 41, wherein said cloned cell or cell line(s) comprise human cells.

50. The method of claim 41, wherein said cloned cell or cell line(s) comprise primary cells in culture.

51. The method of claim 41, wherein said cloned cell or cell line(s) comprise hybridoma cells in culture.

52. The method of claim 41, wherein said cloned cell or cell line(s) is a CHO cell, a CHO cell line, or derived from a CHO cell or CHO cell line.

53. The method of claim 41, wherein said enhanced property is selected from increased efficiency, increased selection rate, increased cell growth, increased appearance speed of selected cells, increased number of selected cell lines, increased doubling time of selected cells, increased cell viability, increased cell line stability, reduced sensitivity to medium depletion and combinations thereof.

54. A method of enhancing the selection rate of semi-stable transfection, comprising the steps of:
(a) culturing a serum-free suspension-adapted Chinese Hamster Ovary (CHO) cell line in glutamine-depleted medium;
(b) mixing said CHO cell line with a DNA composition comprising sequences encoding for a human IL-17F and a glutamine synthase gene;
(c) transporting one or more DNA compositions across the plasma membranes of at least one cell line by electroporation;
(d) culturing transfected cells in said glutamine-depleted medium under selective pressure by adding MSX to the medium; and
(e) allowing transfected cells to express polypeptides encoded by the transfected DNA compositions under selective pressure;
wherein a mixture of cell lines expressing one or more polypeptides is generated that demonstrates an enhanced property of transfection.

55. The method of claim 54, wherein said transfection is stable, and wherein an isolated cell line expressing one or more polypeptides is generated that demonstrates an enhanced property of transfection.

56. The method of claim 55, wherein said method comprises enhancing the selected cell numbers of semi-stable transfection.

57. The method of claim 56, wherein said transfection is stable, and wherein an isolated cell line expressing one or more polypeptides is generated that demonstrates an enhanced property of transfection.
